(19) United States
(12) Patent Application Publication
(10) Pub. No.: US 2024/0000052 A1
(43) Pub. Date: Jan. 4, 2024

(54) TMPRSS KNOCKOUT SWINE HAVING A REDUCED SUSCEPTIBILITY TO INFLUENZA

(71) Applicant: Kansas State University Research Foundation, Manhattan, KS (US)

(72) Inventors: Juergen A. Richt, Manhattan, KS (US); Igor Morozov, Manhattan, KS (US); Bianca Artiaga, Manhattan, KS (US); Taeyong Kwon, Manhattan, KS (US)

(21) Appl. No.: 18/345,951

(22) Filed: Jun. 30, 2023

Related U.S. Application Data
(60) Provisional application No. 63/357,275, filed on Jun. 30, 2022.

(51) Int. Cl. A01K 67/027; C12N 9/76

(52) U.S. Cl. CPC A01K 67/0276; C12N 9/6427; C12Y 304/21004; A01K 2227/108; A01K 2267/02; A01K 2217/075

(57) ABSTRACT

Genetically modified transgenic pigs or pig cells having at least one knocked out Transmembrane protease, serine (TMPRSS) gene. Expression of functional gene products of the at least one knocked out TMPRSS gene in the genetically modified transgenic pig or pig cells is reduced as compared to the non-genetically modified pig or pig cells. The at least one TMPRSS gene can be knocked out using CRISPR/Cas systems.

### CROSS-REFERENCE TO RELATED APPLICATIONS

This application is a non-provisional and claims benefit of U.S. Provisional Application No. 63/357,275 filed Jun. 30, 2022, the specification(s) of which is/are incorporated herein in their entirety by reference.

### FIELD OF THE INVENTION

The present invention relates to the field of genetically modified organisms, for example, genetically modified swine. More specifically, the present invention relates to swine genetically modified for the purpose of making them more resilient to disease, particularly influenza.

### BACKGROUND OF THE INVENTION

Influenza viruses belong to Orthomyxoviridae, a family of negative-sense RNA viruses. This family further comprises seven genera: Alphainfluenzavirus, Betainfluenzavirus, Gammainfluenzavirus, Deltainfluenzavirus, lsavirus, Thogotovirus, and Quaranjavirus, the first four of which are capable of infecting vertebrates. Most significant among these is Alphainfluenzavirus, which includes the Influenza A virus species. Influenza A virus is capable of infecting multiple vertebrate hosts, including humans, swine (pigs), birds, horses, and bats. Influenza A virus is of particular importance to both human and swine hosts. Some influenza A virus subtypes have developed the ability to infect both humans and swine, making swine an important zoonotic origin of human disease. For example, H1N1, the influenza subtype responsible for the 2009 swine flu pandemic, and also believed to have been responsible for the “Spanish Flu” pandemic of 1918, is believed to have resulted from the reassortment of human influenza and swine influenza viruses. H1 indicates that the virus contains hemagglutinin subtype 1 on its surface, and N1 indicates that the virus contains neuraminidase subtype 1 on its surface.

TMPRSS2 (transmembrane serine protease, 2) is crucial for the pathogenesis and infectivity of the influenza virus because the cleavage of influenza virus hemagglutinin by TMPRSS2 facilitates the infection of influenza virus during virus entry into cells. Further, other TMPRSS genes (e.g. TMPRSS7) also exist as part of the TMPRSS subfamily of genes. Pigs are a natural host for influenza viruses and can also act as a “mixing vessel” to generate novel influenza viruses through genetic reassortment, resulting in pandemics with a significant threat to public health and socio-economics, e.g. the swine flu H1N1 pandemic in 2009.

### BRIEF SUMMARY OF THE INVENTION

Provided herein are genetically modified organisms and methods of producing the same. More specifically, the genetically modified organisms may be swine, pigs, or members of the family Suidae, or the genus Sus. In some embodiments, genetically modified pigs with knocked out TMPRSS genes, for example TMPRSS2 and/or TMPRSS4, shed significantly less infectious virus from their nasal cavities upon pH1N2 and H3N2 infections when compared to wild-type pigs. They also have only limited virus replication in their nasal turbinates and less viral replication in the lungs (BALF).

These findings are novel, and contribute to the understanding of the role of various host factors in the life cycle of influenza virus replication and finally provide the basis for the development of potential countermeasures to control influenza virus infection in animals and humans. Furthermore, a commercial use of genetically modified pigs could prevent the economic losses caused by swine influenza virus infection in pigs (˜$5/pig), and the emergence of novel influenza viruses through genetic reassortment.

According to some embodiments, the present invention features a genetically modified transgenic swine comprising at least one modified Transmembrane protease, serine (TMPRSS) gene as compared to a non-genetically modified swine with wild-type TMPRSS gene. Without wishing to limit the present invention to a particular theory or mechanism, expression of functional gene products of the at least one modified TMPRSS gene in the genetically modified transgenic swine is decreased as compared to the non-genetically modified swine. In some embodiments, the swine is less susceptible to an influenza virus and sheds significantly less influenza virus as compared to the non-genetically modified swine.

According to other embodiments, the present invention features a method for making a genetically modified transgenic pig. The method may comprise knocking out (KO) at least one Transmembrane protease, serine (TMPRSS) gene in a pig oocyte; developing the pig oocyte into an embryo; transferring said embryo into a surrogate pig; and gestating in said surrogate pig said embryo into the genetically modified transgenic pig. Without wishing to limit the present invention to a particular theory or mechanism, expression of functional gene products of the at least one KO TMPRSS gene in the genetically modified transgenic pig is decreased as compared to a non-genetically modified pig. In some embodiments, the genetically modified transgenic pig is less susceptible to an influenza virus and sheds significantly less influenza virus as compared to the non-genetically modified pig.

According to some other embodiments, the present invention features a genetically modified transgenic pig cell comprising at least one modified Transmembrane protease, serine (TMPRSS) gene. Without wishing to limit the present invention to a particular theory or mechanism, expression of functional gene products of the at least one modified TMPRSS gene in the genetically modified transgenic pig cell is decreased as compared to the non-genetically modified pig cell. In some embodiments, replication of an influenza virus in the genetically modified transgenic pig cell is impaired as compared to the non-genetically modified pig cell.

In conjunction with the various embodiments of the present invention, the at least one modified TMPRSS gene, e.g. KO TMPRSS gene, may be TMPRSS2, TMPRSS3, TMPRSS4, TMPRSS5, TMPRSS6, TMPRSS7, TMPRSS9, TMPRSS12, TMPRSS13, or TMPRSS15. In some embodiments, the at least one modified TMPRSS gene, e.g. KO TMPRSS gene, has at least one disrupted exon. In other embodiments, at least two TMPRSS genes are modified.

In conjunction with the various embodiments of the present invention, the at least one modified TMPRSS gene, e.g. KO TMPRSS gene, may be a modified TMPRSS2 gene encoding a transmembrane protease, serine 2 having at least 90% amino acid identity corresponding to the wild-type TMPRSS2 gene. In some embodiments, the modified TMPRSS2 gene has a disrupted exon that is any exon other than exon 2. In other embodiments, the modified TMPRSS2 gene has a disrupted Exon 2 and a disrupted Exon 1, or a disrupted Exon 2 and a disrupted Exon 3. In some other embodiments, the at least one modified TMPRSS gene, e.g. KO TMPRSS gene, may be a TMPRSS4 gene encoding a transmembrane protease, serine 4 gene having at least 90% amino acid identity corresponding to the wild-type TMPRSS4 gene.

In conjunction with the various embodiments of the present invention, CRISPR/Cas may be used to knock out the at least one TMPRSS gene. In some embodiments, the TMPRRS2 is knocked out, effectively impairing replication of an influenza virus in the swine as compared to the non-genetically modified swine.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows transmembrane protease, serine 2 (TMPRSS2), a member of the TTSP family and the TMPRSS subfamily, which are integral membrane proteins with an extracellular C-terminal serine protease domain and an N-terminal cytoplasmic domain.

FIG. 2 shows how Hemagglutinin (HA) is synthesized as a precursor (HA₀) that requires cleavage. HA cleavage by membrane-bound proteases can take place in different parts and at different time points during the viral life cycle. HA containing a monobasic cleavage site is cleaved by TMPRSS2 in the Golgi apparatus during assembly or cleaved by HAT on the plasma membrane either during attachment and entry into the cell or during budding of virions.

FIGS. 3A-3E show a plurality of line graphs representing changes in body weight of TMPRSS2 wild-type (+/+), TMPRSS2 heterozygous knockout (+/−), and TMPRSS2 homozygous knockout (−/−) mice infected with various strains of the influenza virus. TMPRSS2-knockout (KO) mice survive infection with an H1N1, H2N1, H7N9, or H10N1 influenza virus that was lethal in wild-type (WT) mice and tend to lose less body weight during the course of infection than wild-type mice. For H3N2 influenza virus, the required protease profile is less defined and appears to be strain-dependent.

FIG. 4 shows the role of TMPRSS2 in activation of Influenza A Virus (IAV) and Influenza B Virus (IBV) with various HAs in murine and human airway cells.

FIG. 5 shows a flow chart of modifying TMPRSS genes in porcine zygotes or embryos and implanting these zygotes or embryos in living pigs.

FIG. 6A shows a genomic locus of a targeted exon.

FIG. 6B shows a location of guides flanking exon 2 of the TMPRSS2 gene. +1 represents the A in the start codon ATG. Guides 1+5, 2+4 and 3+6 were mixed and coinjected. A designed deletion would result in the removal of exon 2 and the start codon.

FIG. 6C shows an example of a zygote being injected with CRIPS/Cas9 RNA.

FIG. 6D shows a photograph of a healthy TMPRSS2 DNA edited pig.

FIG. 6E shows an identification of the products of a polymerase chain reaction of TMPRSS gene product as sorted using agarose gel electrophoresis.

FIGS. 7A-7D show line graphs representing viral titration by TCID₅₀/mL of porcine bronchial epithelial cells (PBEC) collected from TMPRSS2 knockout (KO) and wild-type (WT) control pigs. PBEC cells were established and infected with 0.01 MOI of the pandemic 2009 H1N1 (pH1N1; CA04) or the H3N2 (swine influenza virus TX98) subtypes of influenza A viruses. Infection with the pH1N1 CA04 virus showed a statistical difference between KO and WT cells at 24 h and 36 h post infection, while infection with the H3N2 TX98 showed statistical difference at 24 h, 36 h and 48 h post infection.

FIG. 8A shows a table of the results of influenza virus infection of TMPRSS2 KO pigs and wild pigs.

FIG. 8B shows a timeline of data collection after intratracheal challenge of influenza in both KO and WT pigs.

FIGS. 9A-9B show line graphs of virus nasal shedding after infection of both KO and WT pigs with pH1N1 (FIG. 9A) or H3N2 (FIG. 9B).

FIGS. 10A-10B show histograms of virus replication in nasal turbinates in both KO and WT pigs infected with pH1N1 (FIG. 10A) or H3N2 (FIG. 10B).

FIGS. 11A-11B show histograms of virus loads in bronchoalveolar fluids in both KO and WT pigs infected with pH1N1 (FIG. 11A) or H3N2 (FIG. 11B).

FIGS. 12A-12B show histograms of macroscopic lung pathology in both KO and WT pigs infected with pH1N1 (FIG. 12A) or H3N2 (FIG. 12B).

FIGS. 13A-13E show a timeline of a pH1N1 infection study on KO and WT pigs. One KO pig died of bacterial sepsis on day 2.

FIGS. 14A-14C show results of virus titers in nasal swabs. Virus titers were log-transformed for analysis. Mean±SEM of log (TCID50/ml). Nasal swabs were negative for virus at day 1 and 2. The data of the dead pig was excluded for analysis.

FIGS. 15A-15F show virus titers in tissues and antibody response in both KO and WT pigs infected with pH1N1. The histograms are of virus loads in bronchoalveolar fluids (FIG. 15A), nasal turbinates (FIG. 15B), trachea (FIG. 15C), and lung (FIG. 15D), and macroscopic lung pathology (FIG. 15E) and HI titer (FIG. 15F). The dead KO pig was negative for virus isolation in BALF and three respiratory tissues

FIGS. 16A-16G show a timeline of a pH1N1 transmission study on KO and WT pigs. Both KO and WT pigs infected with pH1N1 were co-mingled with non-infected KO and WT pigs.

FIGS. 17A-17C show virus titers in nasal swabs. Virus titers were log-transformed for analysis. Mean±SEM of log (TCID50/ml).

FIGS. 18A-18F show virus titers in tissues and antibody response in both KO and WT pigs infected with pH1N1. The histograms are of virus loads in nasal turbinates (FIG. 18A), bronchoalveolar fluids (FIG. 18B), trachea (FIG. 18C), and lung (FIG. 18D), and histograms of macroscopic lung pathology (FIG. 18E) and HI titer (FIG. 18F). All BALF and tissues were negative at 21 dpc; <1% lung lesions on 21 dpc.

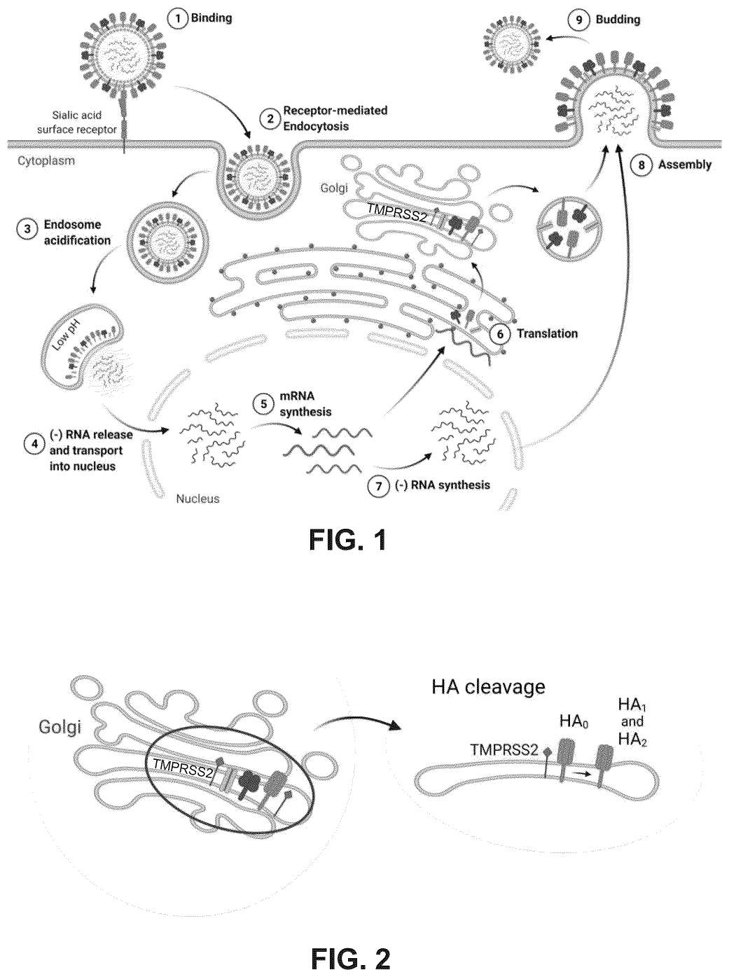

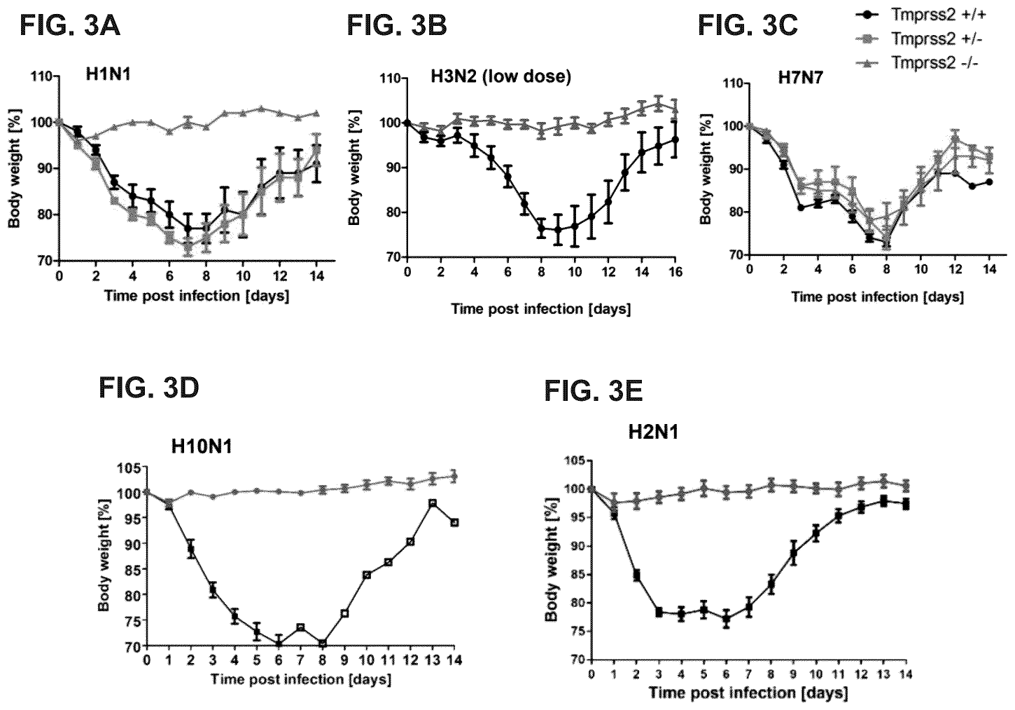

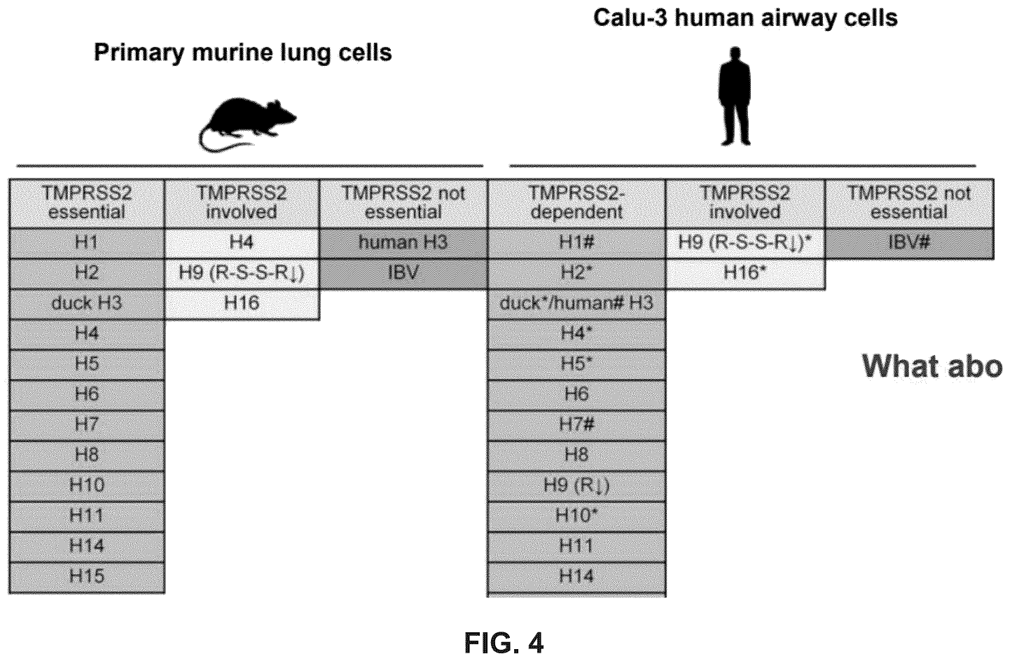

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The practice of some methods disclosed herein employ, unless otherwise indicated, techniques of immunology, biochemistry, chemistry, molecular biology, microbiology, cell biology, genomics and recombinant DNA. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)) (which is entirely incorporated by reference herein).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

A “donor” is meant to include any non-human organism that may serve as a source of donor tissue or cells. The donor may be in any stage of development, including, but not limited to, fetal, neonatal, young and adult. An “animal” is typically a mammal. A “mammal” is meant to include any non-human mammal, including but not limited to pigs, sheep, goats, cattle (bovine), deer, mules, horses, monkeys, dogs, cats, rats, and mice. The term “ungulate” refers to hoofed mammals. Artiodactyla are even-toed (cloven-hooved) ungulates, including antelopes, camels, cows, deer, goats, pigs, and sheep. Perissodactyla are odd toed ungulates, which include horses, zebras, rhinoceroses, and tapirs. The term ungulate as used herein refers to an adult, embryonic or fetal ungulate animal.

As used herein, a “cell” generally refers to a biological cell. A cell may be the basic structural, functional and/or biological unit of a living organism. A cell may originate from any organism having one or more cells, such as a cell from a mammal (e.g., a pig, a cow, a goat, a sheep, a rodent, a rat, a mouse, a non-human primate, a human, etc.).

As used herein, an “organism” may refer to a pig, swine, or a member of the family Suidae, or the genus Sus. For example, a pig can be a wild pig, a domestic pig, mini pigs, a Sus scrofa pig, a Sus scrofa domesticus pig, or inbred pigs. As used herein, the terms “pig” and “swine” and “porcine”, may be used interchangeably to mean any member of the family Suidae, or the genus Sus.

The term “transgene” and its grammatical equivalents as used herein can refer to a gene or genetic material that can be transferred from one organism into another organism. For example, a transgene can be a stretch or segment of DNA containing a gene that is introduced into an organism. When a transgene is transferred into an organism, the organism can then be referred to as a transgenic organism. A transgene can retain its ability to produce RNA or polypeptides (e.g., proteins) in a transgenic organism. A transgene can comprise a polynucleotide encoding a protein or a fragment (e.g., a functional fragment) thereof. The polynucleotide of a transgene can be an exogenous polynucleotide. A fragment (e.g., a functional fragment) of a protein can comprise at least or at least about 5%, 10%, 20%, 30%, 40%, 50%, 60%, 70%, 80%, 90%, 95%, or 99% of the amino acid sequence of the protein. A fragment of a protein can be a functional fragment of the protein. A functional fragment of a protein can retain part or all of the function of the protein.

In one embodiment of the invention, genetically altered pigs and methods of production thereof are provided. The animals of the invention are “genetically modified” or “transgenic,” which means that they have a transgene, or other foreign DNA, added or incorporated, or an endogenous gene modified, including, targeted, recombined, interrupted, deleted, disrupted, replaced, suppressed, enhanced, or otherwise altered, to mediate a genotypic or phenotypic effect in at least one cell of the animal.

In some embodiments, the pig can be at least or at least about 5, 50, 100, or 300 pounds, e.g., the pig can be or be about between 5 pounds to 50 pounds; 25 pounds to 100 pounds; or 75 pounds to 300 pounds.

A genetically modified non-human animal can be of any age. For example, the genetically modified non-human animal can be a fetus; from or from about 1 day to 1 month; from or from about 1 month to 3 months; from or from about 3 months to 6 months; from or from about 6 months to 9 months; from or from about 9 months to 1 year; from or from about 1 year to 2 years. A genetically modified non-human animal can be a non-human fetal animal, perinatal non-human animal, neonatal non-human animal, preweaning non-human animal, young adult non-human animal, or an adult non-human animal.

A genetically modified non-human animal can comprise reduced expression of one or more genes compared to a non-genetically modified counterpart animal, also referred to herein as “wild type” (WT). A non-genetically modified counterpart animal can be an animal substantially identical to the genetically modified animal but without genetic modification in the genome. For example, a non-genetically modified counterpart animal can be a wild-type animal of the same species as the genetically modified animal. The non-human animal can provide cells, tissues or organs for transplanting to a recipient.

The genes whose expression is reduced can include MHC molecules, regulators of MHC molecule expression, and genes differentially expressed between the donor non-human animal and the recipient (e.g., a human or another animal). The reduced expression can be mRNA expression or protein expression of the one or more genes. For example, the reduced expression can be protein expression of the one or more genes. Reduced expression can also include no expression. For example an animal, cell, tissue or organ with reduced expression of a gene can have no expression (e.g., mRNA and/or protein expression) of the gene. Reduction of expression of a gene can inactivate the function of the gene. In some cases, when expression of a gene is reduced in a genetically modified animal, the expression of the gene is absent in the genetically modified animal.

The term “genetic modification”, “gene editing” and their grammatical equivalents as used herein can refer to genetic engineering in which one or more alterations of a nucleic acid, e.g., the nucleic acid within an organism's genome. For example, genetic modification can refer to alterations, additions, and/or deletion of genes. For example, one or more nucleotides being inserted, replaced, or removed from a genome. For example, gene editing can be performed using a nuclease (e.g., a natural-existing nuclease or an artificially engineered nuclease). A genetically modified cell can also refer to a cell with an added, deleted and/or altered gene. A genetically modified cell can be from a genetically modified non-human animal. A genetically modified cell from a genetically modified non-human animal can be a cell isolated from such genetically modified non-human animal. A genetically modified cell from a genetically modified non-human animal can be a cell originated from such genetically modified non-human animal.

The term “expression”, as used herein, generally refers to the process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The term “nucleotide,” as used herein, generally refers to a base-sugar-phosphate combination. A nucleotide may comprise a synthetic nucleotide. A nucleotide may comprise a synthetic nucleotide analog. Nucleotides may be monomeric units of a nucleic acid sequence (e.g., deoxyribonucleic acid (DNA) and ribonucleic acid (RNA)). The term nucleotide may include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof.

The terms “polynucleotide,” “oligonucleotide,” and “nucleic acid” are used interchangeably to generally refer to a polymeric form of nucleotides of any length, either deoxyribonucleotides or ribonucleotides, or analogs thereof, either in single-, double-, or multi-stranded form. A polynucleotide may be exogenous or endogenous to a cell. A polynucleotide may exist in a cell-free environment. A polynucleotide may be a gene or fragment thereof. A polynucleotide may be DNA. A polynucleotide may be RNA. A polynucleotide may have any three-dimensional structure and may perform any function. A polynucleotide may comprise one or more analogs (e.g., altered backbone, sugar, or nucleobase).

The terms “peptide,” “polypeptide,” and “protein” are used interchangeably herein to generally refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer may be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids may include natural amino acids and non-natural amino acids, which have been chemically modified to include a group or a chemical moiety not naturally present on the amino acid. Amino acid analogues may refer to amino acid derivatives. The term “amino acid” includes both D-amino acids and L-amino acids.

The term “promoter”, as used herein, generally refers to the regulatory DNA region which controls transcription or expression of a gene and which may be located adjacent to or overlapping a nucleotide or region of nucleotides at which RNA transcription is initiated. A promoter may contain specific DNA sequences which bind protein factors, often referred to as transcription factors, which facilitate binding of RNA polymerase to the DNA leading to gene transcription. A ‘basal promoter’, also referred to as a ‘core promoter’, may generally refer to a promoter that contains all the basic necessary elements to promote transcriptional expression of an operably linked polynucleotide. Eukaryotic basal promoters typically, though not necessarily, contain a TATA-box and/or a CAAT box.

As used herein, “operably linked”, “operable linkage”, “operatively linked”, or grammatical equivalents thereof generally refers to juxtaposition of genetic elements, e.g., a promoter, an enhancer, a polyadenylation sequence, etc., wherein the elements are in a relationship permitting them to operate in the expected manner. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “vector” as used herein, generally refers to a macromolecule or association of macromolecules that comprises or associates with a polynucleotide and which may be used to mediate delivery of the polynucleotide to a cell. Examples of vectors include plasmids, viral vectors (including baculoviral vectors), liposomes, and other gene delivery vehicles. The vector generally comprises genetic elements, e.g., regulatory elements, operatively linked to a gene to facilitate expression of the gene in a target.

As used herein, a “guide nucleic acid” can generally refer to a nucleic acid that may hybridize to another nucleic acid. A guide nucleic acid may be RNA. A guide nucleic acid may be DNA. The guide nucleic acid may be programmed to bind to a sequence of nucleic acid site-specifically. The nucleic acid to be targeted, or the target nucleic acid, may comprise nucleotides. The guide nucleic acid may comprise nucleotides. A portion of the target nucleic acid may be complementary to a portion of the guide nucleic acid. The strand of a double-stranded target polynucleotide that is complementary to and hybridizes with the guide nucleic acid may be called the complementary strand. The strand of the double-stranded target polynucleotide that is complementary to the complementary strand, and therefore may not be complementary to the guide nucleic acid may be called noncomplementary strand. A guide nucleic acid may comprise a polynucleotide chain and can be called a “single guide nucleic acid.” A guide nucleic acid may comprise two polynucleotide chains and may be called a “double guide nucleic acid.” If not otherwise specified, the term “guide nucleic acid” may be inclusive, referring to both single guide nucleic acids and double guide nucleic acids. A guide nucleic acid may comprise a segment that can be referred to as a “nucleic acid-targeting segment” or a “nucleic acid-targeting sequence.” A nucleic acid-targeting segment may comprise a sub-segment that may be referred to as a “protein binding segment” or “protein binding sequence” or “Cos protein binding segment”.

The terms “complement,” “complements,” “complementary,” and “complementarity,” as used herein, generally refer to a sequence that is fully complementary to and hybridizable to the given sequence. In some cases, a sequence hybridized with a given nucleic acid is referred to as the “complement” or “reverse-complement” of the given molecule if its sequence of bases over a given region is capable of complementarily binding those of its binding partner, such that, for example, A-T, A-U, G-C, and G-U base pairs are formed. In general, a first sequence that is hybridizable to a second sequence is specifically or selectively hybridizable to the second sequence, such that hybridization to the second sequence or set of second sequences is preferred (e.g., thermodynamically more stable under a given set of conditions, such as stringent conditions commonly used in the art) to hybridization with non-target sequences during a hybridization reaction. Typically, hybridizable sequences share a degree of sequence complementarity over all or a portion of their respective lengths, such as between 25%-100% complementarity, including at least 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, and 100% sequence complementarity. Sequence identity, such as for the purpose of assessing percent complementarity, can be measured by any suitable alignment algorithm, including but not limited to the Needleman-Wunsch algorithm (see e.g., the EMBOSS Needle aligner available at www.ebi.ac.uk/Tools/psa/emboss needle/nucleotide.html optionally with default settings), the BLAST algorithm (see e.g., the BLAST alignment tool available at blast.ncbi.nlm.nih.gov/Blast.cgi, optionally with default settings), or the Smith-Waterman algorithm (see e.g., the EMBOSS Water aligner available at www.ebi.ac.uk/Tools/psa/emboss_water/nucleotide.html, optionally with default settings). Optimal alignment can be assessed using any suitable parameters of a chosen algorithm, including default parameters.

The term “percent (%) identity,” as used herein, generally refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment, for purposes of determining percent identity, can be achieved in various ways that are within the skill in the art, for instance, using publicly available computer software such as BLAST, ALIGN, or Megalign (DNASTAR) software. Percent identity of two sequences can be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

As used herein, the term “in vivo” can be used to describe an event that takes place in a subject's body.

As used herein, the term “ex vivo” can be used to describe an event that takes place outside of a subject's body. An “ex vivo” assay cannot be performed on a subject. Rather, it can be performed upon a sample separate from a subject. Ex vivo can be used to describe an event occurring in an intact cell outside a subject's body.

As used herein, the term “in vitro” can be used to describe an event that takes place in a container for holding laboratory reagent(s) such that it is separated from the living biological source organism from which the material is obtained. In vitro assays can encompass cell-based assays in which cells alive or dead are employed. In vitro assays can also encompass a cell-free assay in which no intact cells are employed.

Knocking out technology can comprise gene editing. For example, gene editing can be performed using a nuclease, including CRISPR associated proteins (Cas proteins, e.g., Cas9), Zinc finger nuclease (ZFN), Transcription Activator-Like Effector Nuclease (TALEN), and maganucleases. Nucleases can be naturally existing nucleases, genetically modified, and/or recombinant. For example, a CRISPR/cas system can be suitable as a gene editing system. In some cases, the protein expression of one or more genes is reduced using a CRISPR/cas system.

Knock out (KO) pigs can have decreased expression and decreased protein levels of genes. Overall decreased expression can be less than or less than about 99%, 95%, 90%, 85%, 80%, 75%, 70%, 65%, 60%, 55%, 50%, 45%, 40%, 35%, 30%, 25%, or 20%; e.g., from or from about 99% to 90%; 90% to 80%; 80% to 70%; 70% to 60%; 60% to 50%; 50% to 40%; 40% to 30%, or 30% to 20%, as compared to wild type (WT) pigs that are not knocked out and/or knocked down. Additionally, overall decrease in protein level can be the same as the decreased in overall expression. Overall decrease in protein level can be about or less than about 99%, 95%, 90%, 80%, 70%, 60%, 50%, 40%, 30%, or 20%, e.g., from or from about 99% to 90%; 90% to 80%; 80% to 70%; 70% to 60%; 60% to 50%; 50% to 40%; 40% to 30%, or 30% to 20% as compared to WT pigs.

Knocking out of one or more genes can be validated by genotyping. Methods for genotyping can include sequencing, restriction fragment length polymorphism identification (RFLPI), random amplified polymorphic detection (RAPD), amplified fragment length polymorphism detection (AFLPD), PCR (e.g., long range PCR, or stepwise PCR), allele specific oligonucleotide (ASO) probes, and hybridization to DNA microarrays or beads. For example, genotyping can be performed by sequencing. In some embodiments, sequencing can be high fidelity sequencing or next-generation sequencing. In some embodiments, genotyping can comprise full genome sequencing analysis. In other embodiment, knocking out of a gene in an animal can be validated by sequencing (e.g., next-generation sequencing) a part of the gene or the entire gene.

In a non-limiting embodiment, a CRISPR/Cas system may be used for knocking out a gene. CRISPR/Cas systems are known to those skilled in the art.

For example, double-strand breaks (DSBs) can be generated using a CRISPR/cas system, e.g., a type II CRISPR/cas system. A vector can be operably linked to an enzyme-coding sequence encoding a CRISPR enzyme, such as a Cas protein. Non-limiting examples of Cas proteins include Cas1, Cas1B, Cas2, Cas3, Cas4, Cas5, Cas5d, Cas5t, Cas5h, Cas5a, Cash, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, homologues thereof, or modified versions thereof. A Cas enzyme, such as Cas9, which catalyzes DNA cleavage, can generate double stranded breaks at target site sequences which hybridize to 20 nucleotides of a guide sequence and that have a protospacer-adjacent motif (PAM) following the 20 nucleotides of the target sequence.

A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence. A vector that encodes a CRISPR enzyme that is mutated to with respect, to a corresponding wild-type enzyme such that the mutated CRISPR enzyme lacks the ability to cleave one or both strands of a target polynucleotide containing a target sequence can be used.

As used herein, the term “guide RNA” and its grammatical equivalents can refer to an RNA which can be specific for a target DNA and can form a complex with Cas protein. An RNA/Cas complex can assist in “guiding” Cas protein to a target DNA. A guide RNA can interact with a RNA-guided endonuclease to direct the endonuclease to a specific target site, at which site the 5′ end of the guide RNA base pairs with a specific protospacer sequence in a chromosomal sequence. A guide RNA can comprise two RNAs, e.g., CRISPR RNA (crRNA) and transactivating crRNA (tracrRNA). Furthermore, a crRNA can hybridize with a target DNA. A guide RNA can be an expression product. For example, a DNA that encodes a guide RNA can be a vector comprising a sequence coding for the guide RNA. A guide RNA can be transferred into a cell or organism by transfecting the cell or organism with an isolated guide RNA or plasmid DNA comprising a sequence coding for the guide RNA and a promoter.

A guide RNA can be isolated. A guide RNA can be transferred to a cell in the form of isolated RNA rather than in the form of plasmid comprising encoding sequence for a guide RNA. A guide RNA can comprise three regions: a first region at the 5′ end that can be complementary to a target site in a chromosomal sequence, a second internal region that can form a stem loop structure, and a third 3′ region that can be single-stranded. A first region of each guide RNA can also be different such that each guide RNA guides a fusion protein to a specific target site.

A guide RNA can also be transferred into a cell or organism in other ways, such as using virus-mediated gene delivery. Guide RNA can target a gene in a pig or a pig cell. A guide RNA can be introduced into a cell or embryo as an RNA molecule. For example, a RNA molecule can be transcribed in vitro and/or can be chemically synthesized. An RNA can be transcribed from a synthetic DNA molecule. A guide RNA can then be introduced into a cell or embryo as an RNA molecule. A guide RNA can also be introduced into a cell or embryo in the form of a non-RNA nucleic acid molecule, e.g., DNA molecule. For example, a DNA encoding a guide RNA can be operably linked to promoter control sequence for expression of the guide RNA in a cell or embryo of interest. A RNA coding sequence can be operably linked to a promoter sequence that is recognized by RNA polymerase III (Pol III). A DNA sequence encoding a guide RNA can also be part of a vector. A DNA molecule encoding a guide RNA can also be linear. A DNA molecule encoding a guide RNA can also be circular.

Transformation of mammalian cells can be performed according to standard techniques known in the art. These procedures include the use of viral transduction (for example, by use of any of the viral vectors listed in the preceding paragraph, e.g., by retroviral infection, optionally in the presence of polybrene to enhance infection efficiency), calcium phosphate transfection, electroporation, biolistic particle delivery system (i.e., gene guns), liposomes, microinjection, and any of the other known methods for introducing cloned genomic DNA, cDNA, synthetic DNA or other foreign genetic material into a host cell.

Genetically modified non-human animals disclosed herein can be made using any suitable techniques known in the art. For example, these techniques include, but are not limited to, microinjection, sperm-mediated gene transfer, electroporation of ova or zygotes, and/or nuclear transplantation. The methods of making a non-human animal utilize a cell from a genetically modified non-human animal. For example, a cell can be a somatic cell, such as a fibroblast cell or a fetal fibroblast cell. An enucleated cell can be any cell from an organism. For example, an enucleated cell is a porcine cell. An enucleated cell can be an ovum, for example, an enucleated unfertilized ovum.

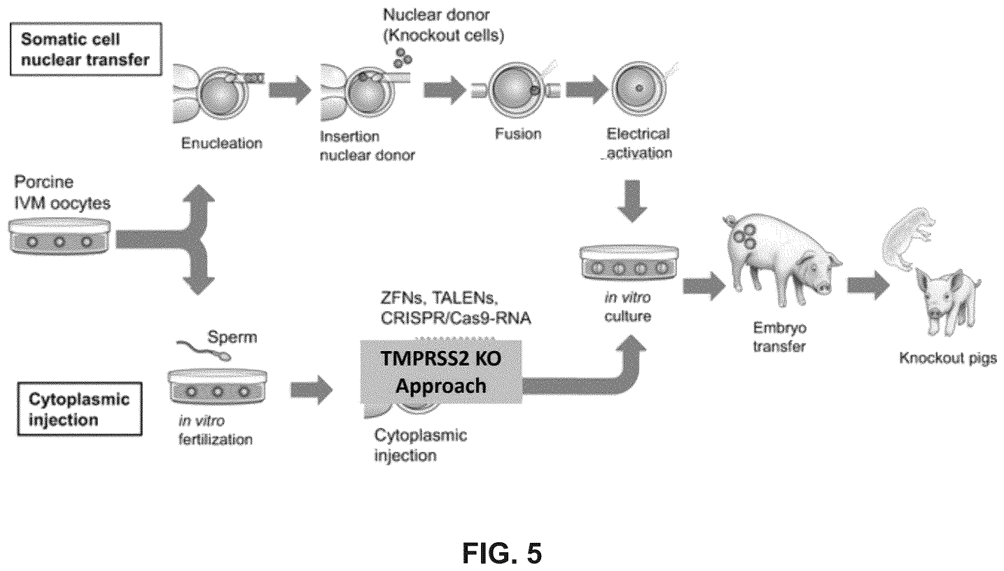

Referring to FIG. 5, in some embodiments, a somatic cell nuclear transfer procedure is utilized. The genome of a somatic porcine cell (e.g., a fetal fibroblast) is genetically modified to create a donor cell. Upon obtaining somatic cells in which a target gene has been targeted, nuclear transfer can be carried out. The genome of a porcine cell (e.g., a fetal fibroblast) is genetically modified by gene targeting as described above, to create a donor cell. The nucleus of such a genetically modified donor cell (or the entire donor cell, including the nucleus) is then transferred into a recipient cell, for example, an enucleated oocyte. The donor cell can be fused with an enucleated oocyte, or donor nucleus or the donor cell itself can be injected into the recipient cell or injected into the perivitelline space, adjacent to the oocyte membrane.

In another embodiment, the genetically modified donor cells can be cryopreserved prior to nuclear transfer. Recipient cells that can be used include oocytes, fertilized zygotes, or the cells of two-cell embryos, all of which may or may not have been enucleated. Recipient oocytes can be obtained using methods that are known in the art or can be purchased from commercial sources. The oocyte can be obtained from a “gilt,” a female pig that has never had offspring or from a “sow,” a female pig that has previously produced offspring.

Cultured cells can be used immediately for nuclear transfer (e.g., somatic cell nuclear transfer), embryo transfer, and/or inducing pregnancy, allowing embryos derived from healthy stable genetic modifications give rise to offspring (e.g., piglets). Such approach can reduce time and cost, e.g., months of costly cell screening that may result in genetically modified cells failing to produce healthy piglets.

Embryo growing and transferring can be performed using standard procedures used in the embryo growing and transfer industry. For example, surrogate mothers can be used. Embryos can also be grown and transferred in culture, for example, by using incubators. In some cases, an embryo can be transferred to an animal, e.g., a surrogate animal, to establish a pregnancy.

The embryos generated herein can be transferred to surrogate non-human animals (e.g., pigs) to produce offspring (e.g., piglets). For example, the embryos can be transferred to the oviduct of recipient gilts. Pregnancy can be diagnosed, e.g., by ultrasound. Pregnancy can be diagnosed after or after about 28 days from the transfer . . . . All of the microinjected offspring (e.g., piglets) can be delivered by natural birth. Information of the pregnancy and delivery (e.g., time of pregnancy, rates of pregnancy, number of offspring, survival rate, etc.) can be documented. The genotypes and phenotypes of the offspring can be measured using any methods described through the application such as sequencing (e.g., next-generation sequencing).

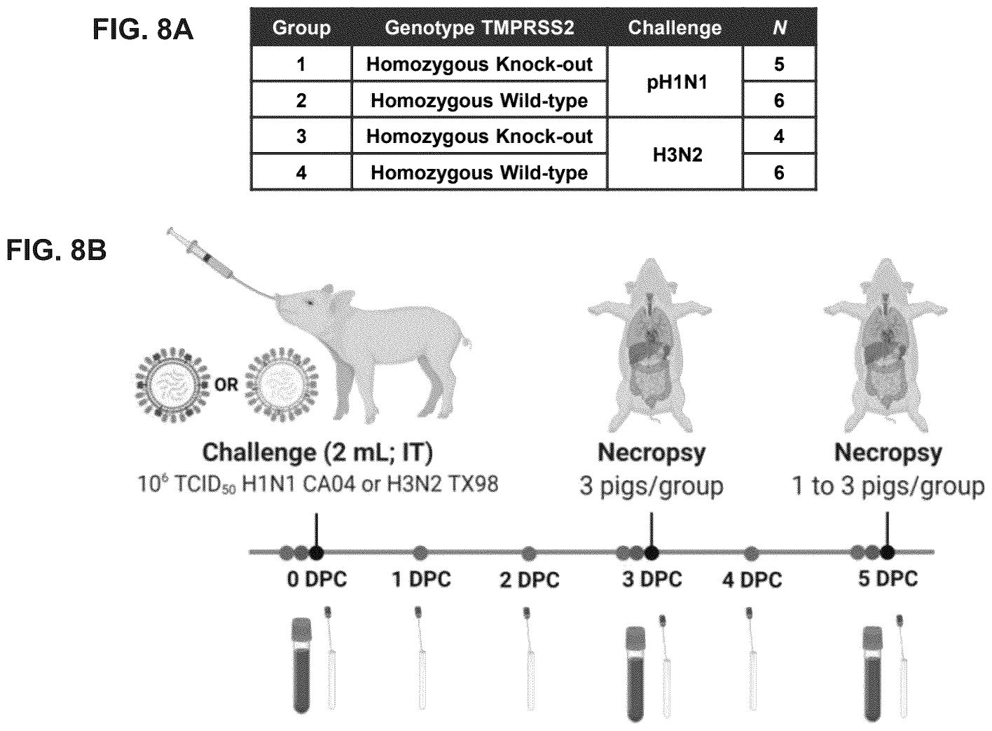

In some embodiments, the present invention features a knockout swine comprising at least two modified TMPRSS (Transmembrane protease, serine) genes as compared to a swine with wild-type TMPRSS genes. For example, the modified TMPRSS genes may have decreased expression of functional gene products. The functional gene products may be, for example, enzymes. In some embodiments, at least two TMPRSS genes are modified in the same knockout swine. For example, a knockout swine may have two modified TMPRSS genes, each of which has a decreased expression of functional enzyme as compared to a wild-type swine. In some embodiments, the modified TMPRSS genes may be homozygous knockouts. See FIG. 8A. For example, both diploid copies of the TMPRSS4 gene may be knocked out. In some embodiments, the modified TMPRSS genes may be heterozygous knockouts. See FIG. 8A. For example, a single diploid copy of the TMPRSS5 gene may be knocked out.

With respect to TMPRSS genes, pigs (Sus scrofa, Sus scrofa domesticus, etc.) have 10 different TMPRSS genes belonging to the overarching TMPRSS gene subfamily: TMPRSS2, TMPRSS3, TMPRSS4, TMPRSS5, TMPRSS6, TMPRSS7, TMPRSS9, TMPRSS12, TMPRSS13, and TMPRSS15. See Table 1, below.

TABLE 1
Sus scrofa TMPRSS Genes Belonging to the TMPRSS Gene Family

| | | | | | | |
|---|---|---|---|---|---|---|
| | | | Vertebrate Gene | | | |
| | | | Nomenclature | | Number | |
| TMPRSS | Accession | | Committee | | of | |
| Gene | Number(s) | GenBank ID | (VGNC) Number | Ensembl ID | Exons | GenBank Link |

| | | | | | |
|---|---|---|---|---|---|
| TMPRSS1 | | | | | |
| TMPRSS2 | 100739292 | 94234 | ENSSSCG00000024336 | 14 | http://view.ncbi.nlm.nih.gov/gene/100739292 |
| TMPRSS3 | 100627826 | 94235 | ENSSSCG00000022568 | 14 | https://www.ncbi.nlm.nih.gov/gene/100627826 |
| TMPRSS4 | 100514419 | 94236 | ENSSSCG00000015086 | 13 | https://www.ncbi.nlm.nih.gov/gene/100514419 |
| TMPRSS5 | 100515912 | 94237 | ENSSSCG00000015049 | 13 | https://www.ncbi.nlm.nih.gov/gene/100515912 |
| TMPRSS6 | 100516673 | 94238 | ENSSSCG00000026067 | 18 | https://www.ncbi.nlm.nih.gov/gene/100516673 |
| TMPRSS7 | 100627225 | 94239 | ENSSSCG00000025721 | 18 | https://www.ncbi.nlm.nih.gov/gene/100627225 |
| TMPRSS8 | | | | | |
| TMPRSS9 | 100621666 | 94240 | ENSSSCG00000028363 | 19 | https://www.ncbi.nlm.nih.gov/gene/100621666 |
| TMPRSS10 | | | | | |
| TMPRSS11 | | | | | |
| TMPRSS12 | 110260566 | 94231 | ENSSSCG00000034783 | 5 | https://www.ncbi.nlm.nih.gov/gene/110260566 |
| TMPRSS13 | 100511523 | 94232 | ENSSSCG00000038710 | 14 | https://www.ncbi.nlm.nih.gov/gene/100511523 |
| TMPRSS14 | | | | | |
| TMPRSS15 | 397152 | 94233 | ENSSSCG00000012019 | 27 | https://www.ncbi.nlm.nih.gov/gene/397152 |
| TMPRSS16 | | | | | |

Each of these genes has varying numbers of exons. For example, TMPRSS2 has 14 exons, TMPRSS6 has 18 exons, TMPRSS9 has 19 exons, and TMPRSS15 has 27 exons. See Table 2a/b/c/d, below. These tables show various TMPRSS variants and exon numbers within each variant. The present invention may encompass knockout swine comprising knockouts of various combinations of exons within various combinations of TMPRSS variants as described in these tables. The “X” represents possible exons that may be disrupted in order to modify the respective wild-type TMPRSS genes as listed above.

These exons may be disrupted via genetic modification techniques, such as CRISPR/Cas, transcription activator-like effector nucleases (TALENs), zinc finger nucleases (ZFN), or other appropriate methods. See FIG. 5. For additional details regarding modification techniques, see Appendix A, the disclosures of which are incorporated in their entirety by reference herein.

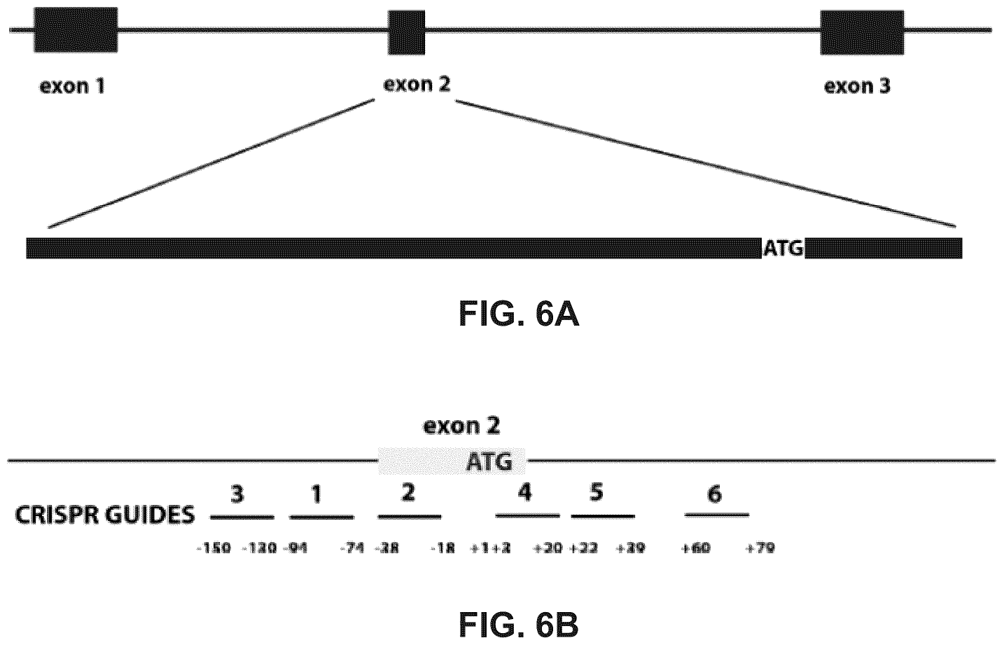

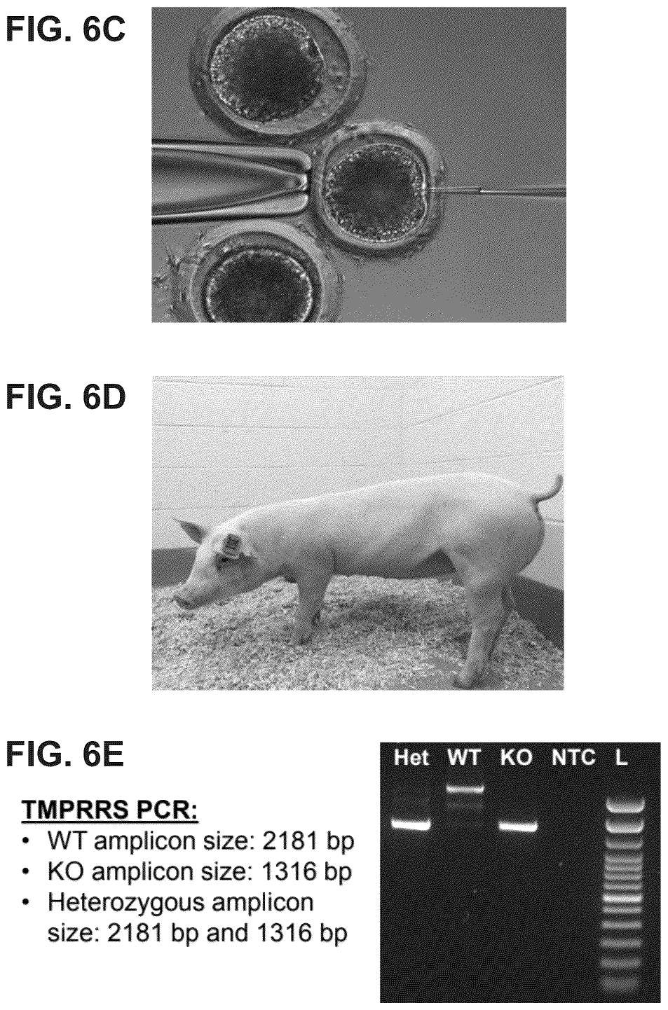

In some embodiments, these genetic modification techniques may be performed on a zygote. See FIG. 6C. In some embodiments, disruption of these exons may result in truncated genes or truncated gene products. See FIG. 6E. Disruption of these exons may reduce the function of the functional gene products of these various TMPRSS enzymes. For example, disruption of exon 5 of TMPRSS2 may result in a TMPRSS2 gene product (i.e., TMPRSS2 enzyme) with reduced enzymatic activity. For example, a TMPRSS2 enzyme produced by a TMPRSS2 gene with a disrupted exon 5 may have reduced function as compared to a TMPRSS2 enzyme produced by a wild-type TMPRSS2 gene. For example, the TMPRSS2 enzyme produced from a TMPRSS2 gene with a disrupted exon 5 may have reduced and/or altered function because it has reduced and/or altered expression (i.e., may be reduced in quantity), has reduced and/or altered enzymatic activity (i.e., may have reduced catalytic activity), has reduced and/or altered function for other reasons, or may have reduced and/or altered function for a combination of different reasons.

Tables 2a-2d show various TMPRSS variants and exon numbers within each variant. The present invention may encompass knockout swine comprising knockouts of various combinations of exons within the various combinations of TMPRSS variants as described in this table. Key: “X” represents possible exons that may be disrupted in order to modify the respective wild-type TMPRSS genes as listed below.

TABLE 2a

| | Exon | Exon | Exon | Exon | Exon | Exon | Exon | Exon | Exon | Exon | Exon | Exon | Exon |
|---|---|---|---|---|---|---|---|---|---|---|---|---|---|
| Gene | 1 | 2 | 3 | 4 | 5 | 6 | 7 | 8 | 9 | 10 | 11 | 12 | 13 |
| TMPRSS1 | | | | | | | | | | | | | |
| TMPRSS2 | X | X | X | X | X | X | X | X | X | X | X | X | X |
| TMPRSS3 | X | X | X | X | X | X | X | X | X | X | X | X | X |
| TMPRSS4 | X | X | X | X | X | X | X | X | X | X | X | X | X |
| TMPRSS5 | X | X | X | X | X | X | X | X | X | X | X | X | X |
| TMPRSS6 | X | X | X | X | X | X | X | X | X | X | X | X | X |
| TMPRSS7 | X | X | X | X | X | X | X | X | X | X | X | X | X |
| TMPRSS8 | | | | | | | | | | | | | |

TABLE 2b

| | Exon | Exon | Exon | Exon | Exon | Exon | Exon | Exon | Exon | Exon | Exon | Exon | Exon | Exon |
|---|---|---|---|---|---|---|---|---|---|---|---|---|---|---|
| Gene | 14 | 15 | 16 | 17 | 18 | 19 | 20 | 21 | 22 | 23 | 24 | 25 | 26 | 27 |

| | | | | | |
|---|---|---|---|---|---|
| TMPRSS1 | | | | | |
| TMPRSS2 | X | | | | |
| TMPRSS3 | X | | | | |
| TMPRSS4 | | | | | |
| TMPRSS5 | | | | | |
| TMPRSS6 | X | X | X | X | X |
| TMPRSS7 | X | X | X | X | X |
| TMPRSS8 | | | | | |

TABLE 2c

| | Exon | Exon | Exon | Exon | Exon | Exon | Exon | Exon | Exon | Exon | Exon | Exon | Exon |
|---|---|---|---|---|---|---|---|---|---|---|---|---|---|
| Gene | 1 | 2 | 3 | 4 | 5 | 6 | 7 | 8 | 9 | 10 | 11 | 12 | 13 |
| TMPRSS9 | X | X | X | X | X | X | X | X | X | X | X | X | X |
| TMPRSS10 | | | | | | | | | | | | | |
| TMPRSS11 | | | | | | | | | | | | | |
| TMPRSS12 | X | X | X | X | X | | | | | | | | |
| TMPRSS13 | X | X | X | X | X | X | X | X | X | X | X | X | X |
| TMPRSS14 | | | | | | | | | | | | | |
| TMPRSS15 | X | X | X | X | X | X | X | X | X | X | X | X | X |

TABLE 2d

| | Exon | Exon | Exon | Exon | Exon | Exon | Exon | Exon | Exon | Exon | Exon | Exon | Exon | Exon |
|---|---|---|---|---|---|---|---|---|---|---|---|---|---|---|
| Gene | 14 | 15 | 16 | 17 | 18 | 19 | 20 | 21 | 22 | 23 | 24 | 25 | 26 | 27 |
| TMPRSS9 | X | X | X | X | X | X | | | | | | | | |
| TMPRSS10 | | | | | | | | | | | | | | |
| TMPRSS11 | | | | | | | | | | | | | | |
| TMPRSS12 | | | | | | | | | | | | | | |
| TMPRSS13 | X | | | | | | | | | | | | | |
| TMPRSS14 | | | | | | | | | | | | | | |
| TMPRSS15 | X | X | X | X | X | X | X | X | X | X | X | X | X | X |

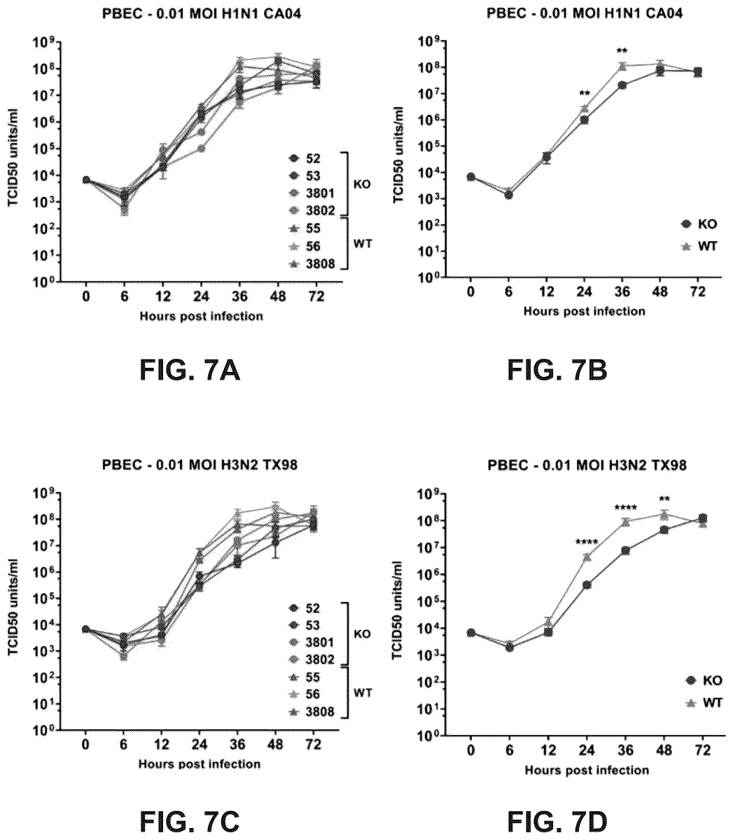

In some embodiments, the reduction in the function of TMPRSS genes may result in an organism (e.g., a pig) having a decreased susceptibility to viral infection. For example, a pig with a modified TMPRSS7 gene (featuring, for example, a deletion of exon 13 of TMPRSS7) may have a decreased susceptibility to viral infection. In some embodiments, this decreased susceptibility to viral infection may manifest in a reduced susceptibility to infection with an influenza virus. For example, a pig with a modified TMPRSS9 gene may have a decreased susceptibility to infection with an influenza virus. In some embodiments, this virus may be the pH1N1 subtype of influenza viruses. In some embodiments, this virus may be the H1N1 CA04 subtype of influenza viruses. See FIGS. 7A and 7B. In some embodiments, this virus may be the H3N2 subtype of influenza viruses. In some embodiments, this virus may be the H3N2 TX98 subtype of influenza viruses. See FIGS. 7C and 7D. In some embodiments, this virus may be the H1N1 subtype of influenza viruses. In some embodiments, this virus may be the H7N7 subtype of influenza viruses. In some embodiments, this virus may be the H10N1 subtype of influenza viruses. In some embodiments, this virus may be the H2N1 subtype of influenza viruses. In some embodiments, this virus may be other subtypes or variants of the influenza virus. In some embodiments, this virus may be a virus other than the influenza virus. In some embodiments, modified TMPRSS genes may result in a decreased susceptibility to other types of infection, e.g. bacterial infection.

In some embodiments, the modification of TMPRSS genes may result in an organism with decreased susceptibility to viral infection in the form of altered viral growth kinetics in the organism's cells. For example, modified TMPRSS genes may result in altered viral growth kinetics in the organism's bronchial epithelial cells. See FIGS. 7A-7D. For example, pigs with a modified TMPRSS6 gene may have delayed or diminished viral growth in their bronchial epithelial cells.

In some embodiments, modification of TMPRSS genes may result in an organism exhibiting reduced weight loss following influenza infection as compared to a wild-type organism.

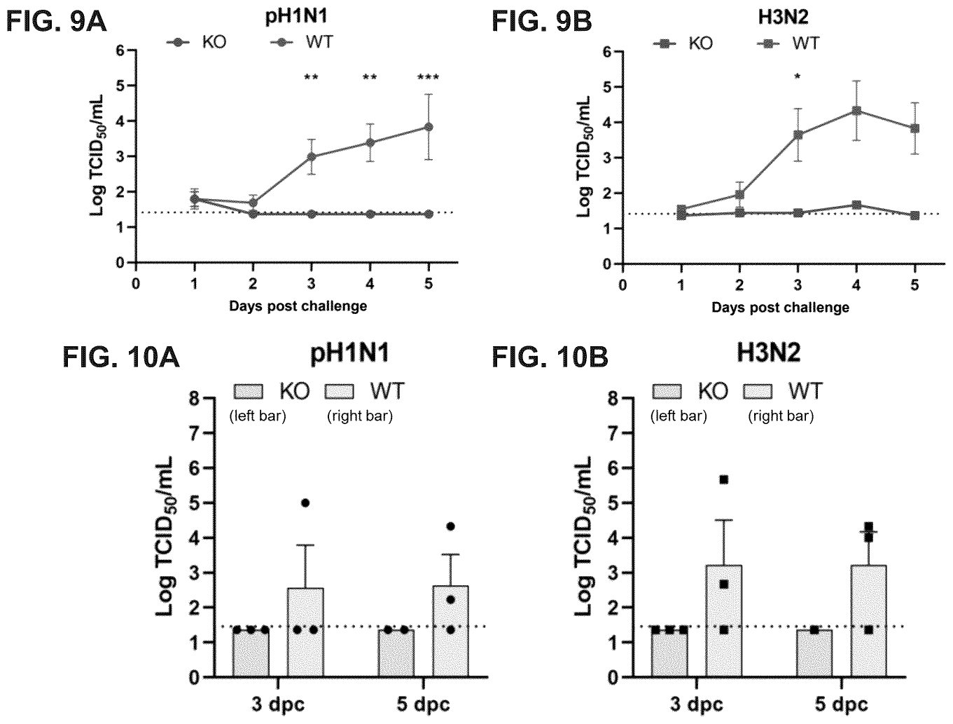

In some embodiments, the modification of TMPRSS genes may result in an organism with decreased susceptibility to viral infection in the form of decreased nasal shedding of virus after infection. See FIGS. 9A and 9B. For example, pigs with a modified TMPRSS13 gene may have decreased nasal shedding of virus after infection. In some embodiments this may reduce the infectivity of the infected host organism. This may also result in decreased symptoms in the infected host. This may also result in decreased mortality and/or morbidity in the infected host.

In some embodiments, the modification of TMPRSS genes may result in an organism with decreased susceptibility to viral infection in the form of decreased virus replication in the nasal turbinates of the organism after infection. See FIGS. 10A and 10B. For example, pigs with a modified TMPRSS12 gene may have decreased virus replication in their nasal turbinates after infection. In some embodiments this may reduce the infectivity of the infected host organism. This may also result in decreased symptoms in the infected host. This may also result in decreased mortality or morbidity in the infected host.

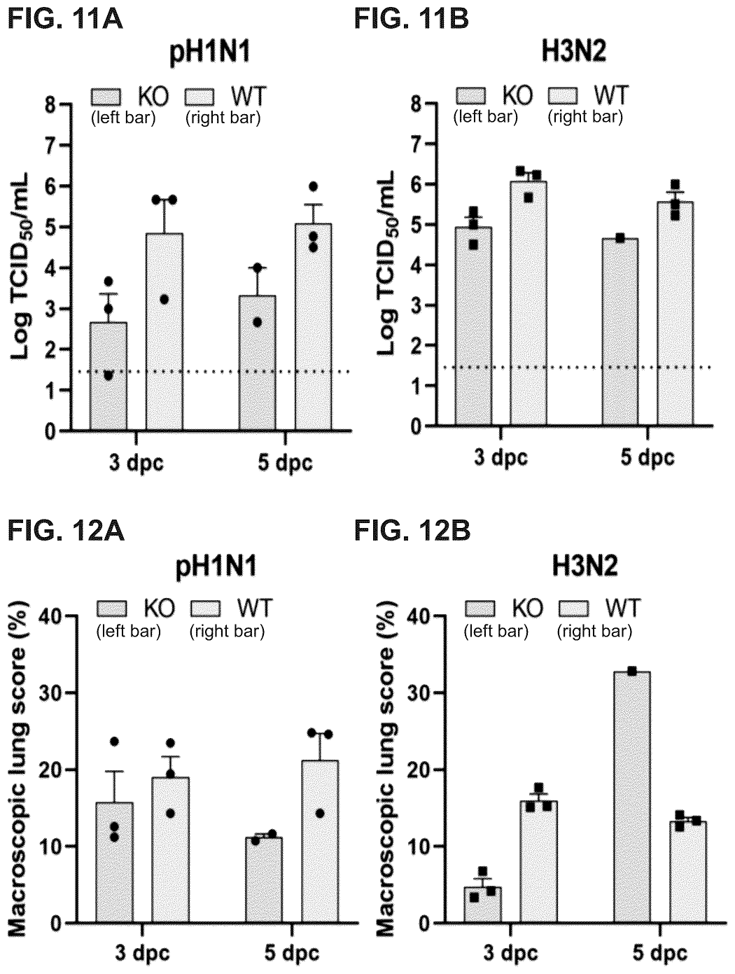

In some embodiments, the modification of TMPRSS genes may result in an organism with decreased susceptibility to viral infection in the form of decreased virus loads in the bronchoalveolar fluids of the organism after infection. See FIGS. 11A and 11B. For example, pigs with a modified TMPRSS7 gene may have decreased virus loads in their bronchoalveolar fluids after infection. This may reduce the infectivity of the infected host organism. This may also result in decreased symptoms in the infected host. This may also result in decreased mortality or morbidity in the infected host.

In some embodiments, the modification of TMPRSS genes may result in an organism with decreased susceptibility to viral infection in the form of decreased lung pathology in the organism after infection. See FIGS. 12A and 12B. For example, pigs with a modified TMPRSS3 gene may have less severe lung pathology after infection. This may reduce the infectivity of the infected host organism. This may also result in decreased symptoms in the infected host. This may also result in decreased mortality or morbidity in the infected host.

In some embodiments, the present invention features a knockout swine comprising a modified TMPRSS (Transmembrane protease, serine) gene as compared to a swine with wild-type TMPRSS genes, wherein expression of functional gene products of the modified TMPRSS gene in the knockout swine is decreased as compared to a wild-type swine. In some embodiments, the knockout swine may comprise a modified TMPRSS2 gene as compared to a swine with a wild-type TMPRSS2 gene. In some embodiments, the knockout swine may comprise a modified TMPRSS2 gene and a modified TMPRSS3 gene as compared to a swine with a wild-type TMPRSS2 gene and a wild-type TMPRSS3 gene. The modified TMPRSS2 gene may be modified by featuring a disrupted exon 2. The modified TMPRSS2 gene may be modified by featuring a disrupted exon 4. The modified TMPRSS2 gene may be modified by featuring a disrupted exon 11. See Table 2a, Table 2b, Table 2c, and Table 2d, above. The present invention is not limited to these examples, and may comprise modifications to any combination of the various TMPRSS genes and disruption and/or other modification of their corresponding exons and/or other regions, as selected from any of the options in Table 2a, Table 2b, Table 2c, and Table 2d.

In some embodiments, the knockout swine may comprise a modified TMPRSS7 gene as compared to a swine with a wild-type TMPRSS7 gene. The modified TMPRSS7 gene may be modified by featuring a disrupted exon 5. The modified TMPRSS7 gene may be modified by featuring a disrupted exon 13. The modified TMPRSS7 gene may be modified by featuring a disrupted exon 17. See Table 2a, Table 2b, Table 2c, and Table 2d, above. The present invention is not limited to these examples, and may comprise modifications to any combination of the various TMPRSS genes and disruption and/or other modification of their corresponding exons and/or other regions, as selected from any of the options in Table 2a, Table 2b, Table 2c, and Table 2d.

In some embodiments, the knockout swine may comprise a modified TMPRSS2 (Transmembrane protease, serine 2) gene as compared to a swine with a wild-type TMPRSS2 gene. In some embodiments, expression of functional transmembrane protease, serine 2 in the knockout swine is decreased as compared to a wild-type swine. In some embodiments, the TMPRSS2 gene is modified at at least two separate regions of the TMPRSS2 gene.

In some embodiments, the knockout swine may comprise a modified TMPRSS2 gene as compared to a swine with a wild-type TMPRSS2 gene. The modified TMPRSS2 gene may be modified by featuring disruptions or other modifications to at least two separate regions of the TMPRSS2 gene. The modified TMPRSS2 gene may be modified by featuring disruptions or other modifications to, for example, exon 5 and exon 8. The modified TMPRSS2 gene may be modified by featuring disruptions or other modifications to, for example, exon 9 and exon 12. The modified TMPRSS2 gene may be modified by featuring disruptions or other modifications to, for example, exon 1, exon 7, and exon 10. The modified TMPRSS2 gene may be modified by featuring disruptions or other modifications to, for example, exon 3, exon 6, exon 8, and exon 14. The present invention is not limited to these examples, and may comprise disruption and/or other modifications to any combination of the various at least two regions and/or exons of TMPRSS2, as selected from any of the options in Table 2a and Table 2b, above.

In some embodiments, the knockout swine may comprise a knockout swine comprising a modified TMPRSS2 (Transmembrane protease, serine 2) gene as compared to a swine with a wild-type TMPRSS2 gene, wherein expression of functional transmembrane protease, serine 2 in the knockout swine is decreased as compared to a wild-type swine and wherein the modified TMPRSS2 gene has a disrupted exon that is any exon other than exon 2.

In some embodiments, the knockout swine may comprise a modified TMPRSS2 gene as compared to a swine with a wild-type TMPRSS2 gene. The modified TMPRSS2 gene may be modified by featuring disruptions or other modifications to any exon other than exon 2. The modified TMPRSS2 gene may be modified by featuring disruptions to, for example, exon 1. The modified TMPRSS2 gene may be modified by featuring disruptions to, for example, exon 3. The modified TMPRSS2 gene may be modified by featuring disruptions to, for example, exon 5. The modified TMPRSS2 gene may be modified by featuring disruptions to, for example, exon 9. The modified TMPRSS2 gene may be modified by featuring disruptions to, for example, exon 13. The modified TMPRSS2 gene may be modified by featuring disruptions to, for example, exon 14. The modified TMPRSS2 gene may be modified by featuring disruptions to, for example, exon 3 and exon 9. The modified TMPRSS2 gene may be modified by featuring disruptions to, for example, exon 5, exon 11, and exon 12. The modified TMPRSS2 gene may be modified by featuring disruptions to, for example, exon 1, exon 7, exon 11, and exon 13. The present invention is not limited to these examples, and may comprise disruption of any TMPRSS2 exon other than exon 2, or disruption of any combination of the TMPRSS2 exons, provided that exon 2 is not disrupted, as selected from any of the options in Table 2a and Table 2b, above.

In some embodiments the knockout swine may comprise a modified TMPRSS2 (Transmembrane protease, serine 2) gene as compared to a swine with wild-type TMPRSS2 gene, and a modified TMPRSS4 (Transmembrane protease, serine 4) gene as compared to a swine with a wild-type TMPRSS4 gene, wherein expression of functional gene products of both the modified TMPRSS2 gene and the modified TMPRSS4 gene in the knockout swine is decreased as compared to a wild-type swine.

In some embodiments, the knockout swine may comprise a modified TMPRSS2 gene as compared to a swine with a wild-type TMPRSS2 gene and a modified TMPRSS4 gene as compared to a swine with a wild-type TMPRSS4 gene. The modified TMPRSS2 gene may be modified by featuring disruptions to, for example, exon 4. The modified TMPRSS2 gene may be modified by featuring disruptions to, for example, exon 7. The modified TMPRSS2 gene may be modified by featuring disruptions to, for example, exon 1 and exon 11. The modified TMPRSS2 gene may be modified by featuring disruptions to, for example, exon 3, exon 4, and exon 12. The modified TMPRSS4 gene may be modified by featuring disruptions to, for example, exon 5. The modified TMPRSS4 gene may be modified by featuring disruptions to, for example, exon 11. The modified TMPRSS4 gene may be modified by featuring disruptions to, for example, exon 3 and exon 13. The modified TMPRSS4 gene may be modified by featuring disruptions to, for example, exon 6, exon 9, and exon 11. The present invention is not limited to these examples, and may comprise any combination of modified TMPRSS2 and modified TMPRSS4, wherein such modification can occur through various means, including but not limited to disruption and/or other modification of various combinations of the exons and/or other regions of each TMPRSS2 and TMPRSS4, as listed in Table 2a and Table 2b, above.

Details of how the knockout swine were made can be found in U.S. Ser. No. 10/091,975B2, the specification of which is incorporated herein by reference in its entirety. Further details of how the knockout swine were made can also be found in Appendix A, which contains the following document: Whitworth, K. M., Benne, J. A., Spate, L. D. et al. Zygote injection of CRISPR/Cas9 RNA successfully modifies the target gene without delaying blastocyst development or altering the sex ratio in pigs. Transgenic Res 26, 97-107 (2017). https://doi.org/10.1007/s11248-016-9989-6. The contents of said document in Appendix A are incorporated herein by reference in its entirety. Appendix A forms part of the Specification and is part of the original disclosure.

### EXAMPLE

The following is a non-limiting example of the present invention. It is to be understood that said example is not intended to limit the present invention in any way. Equivalents or substitutes are within the scope of the present invention.

### Example 1

In Vitro Studies: In order to determine the effect of TMPRSS2 on influenza virus replication in vitro, primary cells were harvested from TMPRSS2 knockout (KO) and wild-type (WT) control pigs and infected with the pH1N1 and the Tx/98 H3N2 subtypes of influenza viruses (FIGS. 7A-7D). Collection and establishment of primary bronchial cells from KO and WT piglets for in vitro studies was performed. Infection with H1N1 CA04 showed a statistical difference between KO and WT cells at 24 h and 36 h post-infection. Infection with H3N2 TX98 also showed a statistical difference between KO and WT cells at 24 h, 36 h and 48 h.

In Vivo Studies: TMPRSS2 KO (n=5 [pH1N1] or n=4 [H3N2]) and WT (n=6) pigs were infected with either pH1N1 or H3N2 subtypes of the influenza virus in order to characterize viral replication in vivo. Clinical samples were collected during the course of infection, and tissue samples were obtained at necropsy. Virus titers in samples were determined by endpoint titration.

Nasal Swabs: In nasal swabs of pH1N1-infected KO pigs (FIGS. 9A-9B), the influenza virus was only isolated at 1-day post-challenge (dpc), but no virus was isolated between 2 and 5 dpc. The virus titers in nasal swab of WT pigs continued to increase from day 1 to 5 dpc. There were significant differences of nasal shedding at 3, 4, and 5 dpc in this group. Similarly, viral shedding in H3N2-infected KO pigs was reduced compared to the WT pigs, and a statistical difference was found at 3 dpc.

Nasal Turbinates: None of KO pigs were positive for virus isolation at 3 and 5 dpc in nasal turbinates of pH1N1-infected pigs (FIGS. 10A-10B). In contrast, the virus was isolated from one of three WT pigs at 3 dpc and two of three at 5 dpc. In addition, no virus was isolated from nasal turbinates of H3N2-infected KO pigs; in contrast, two out of three H3N2-infected WT pigs had infectious viruses in nasal turbinates at 3 dpc and 5 dpc.

Bronchioalveolar Lavage Fluid (BALF): In the pH1N1-infected group (FIGS. 11A-11B), the virus titer in the bronchioalveolar lavage fluid (BALF) of KO pigs was lower at 3 and 5 dpc than in the WT pigs. In the H3N2-infected group, the KO pigs also had lower virus titers in the BALF at 3 dpc and 5 dpc.

Macroscopic Pathology: Infection of KO pigs with pH1N1 resulted in less severe lung lesions at 5 dpc, and with H3N2 at 3 dpc compared to WT pigs whereas on 5 dpc, the KO pigs had more severe lung lesions compared to WT pigs (FIGS. 12A-12B).

### Example 2

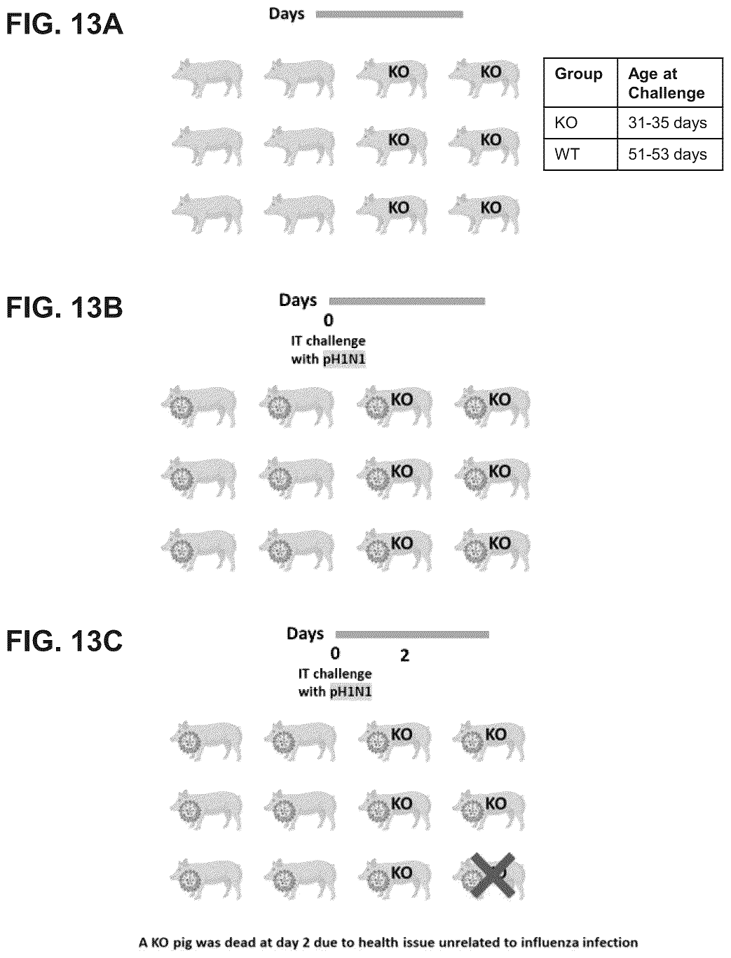

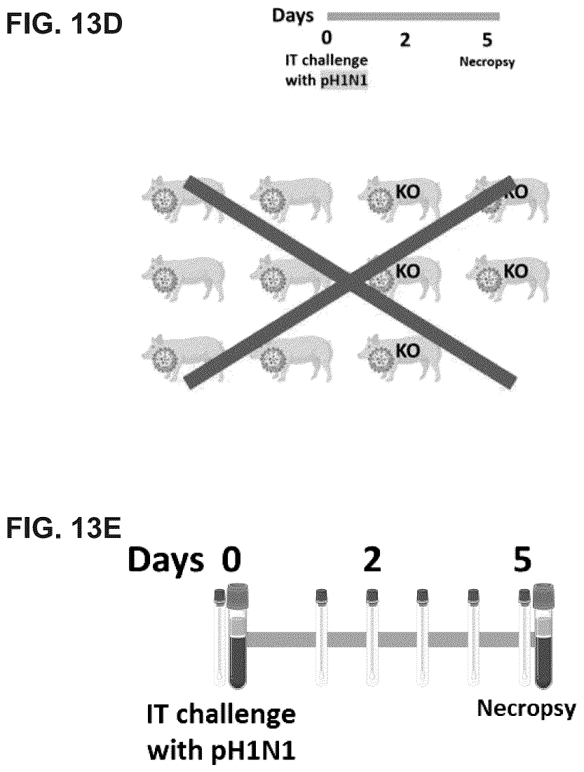

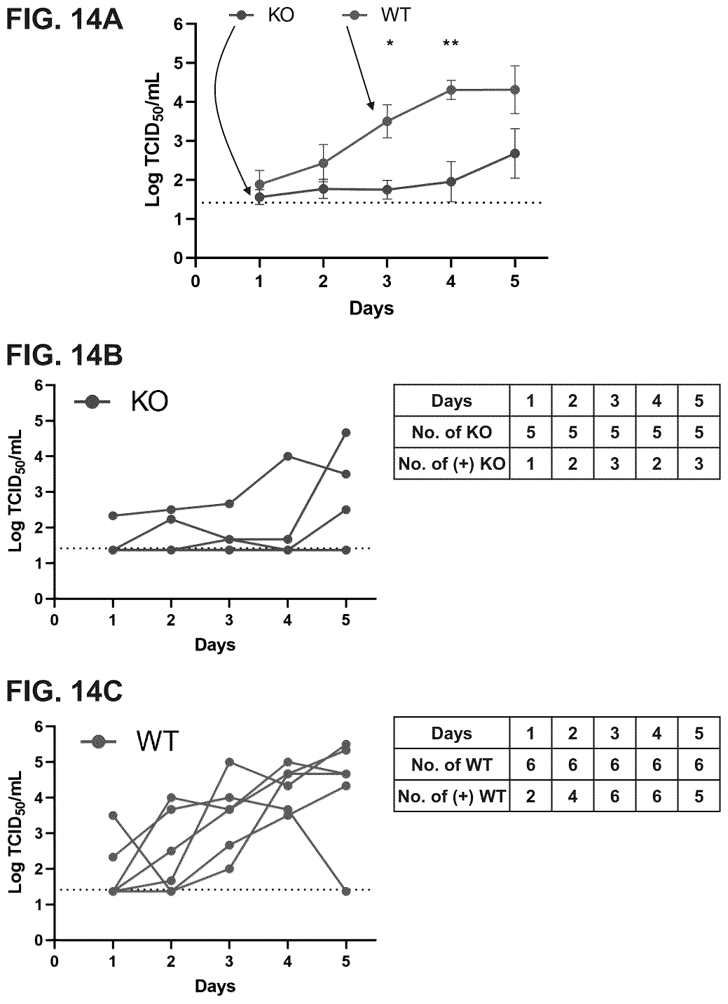

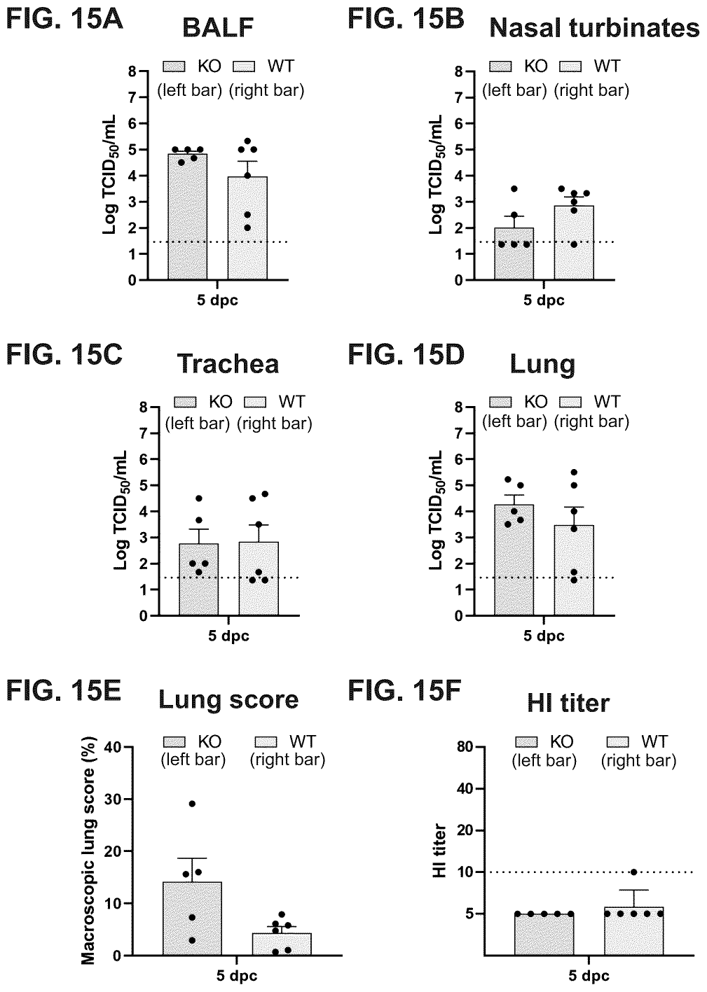

Referring to FIGS. 13A-15F, in the susceptibility study, it was confirmed that TMPRSS2 KO pigs shed significantly less influenza virus in nasal swabs compared to WT controls after influenza virus infection.

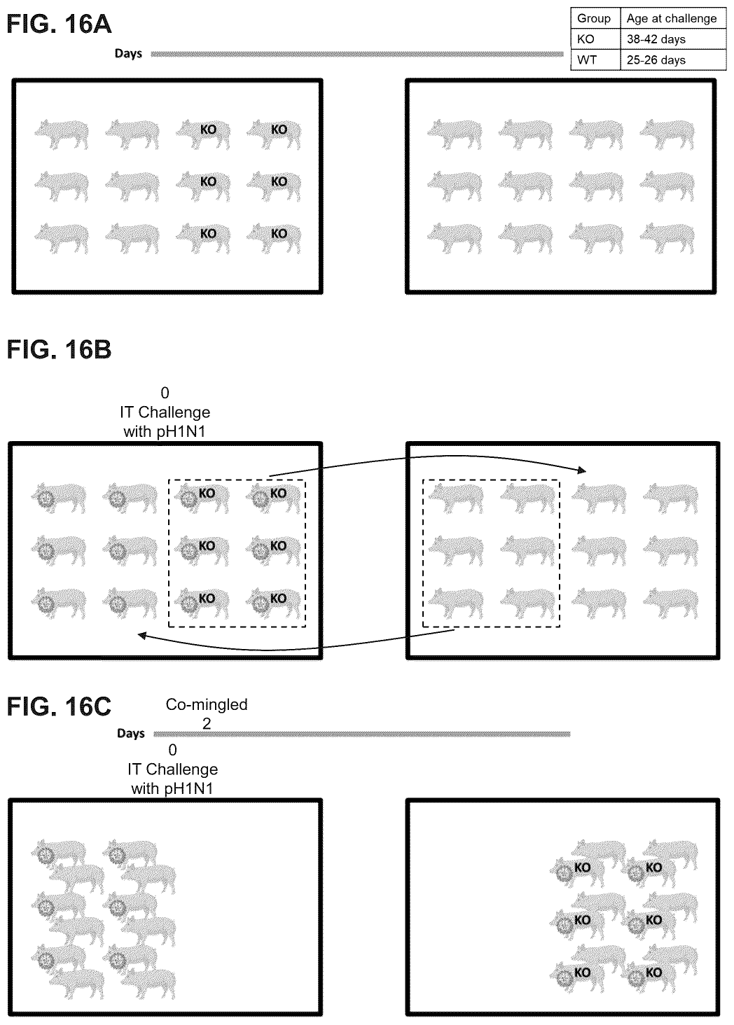

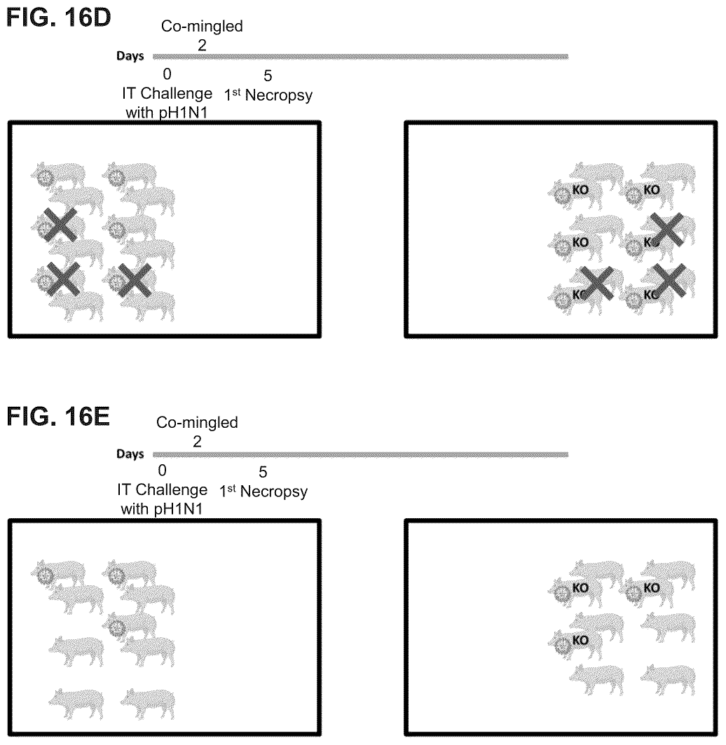

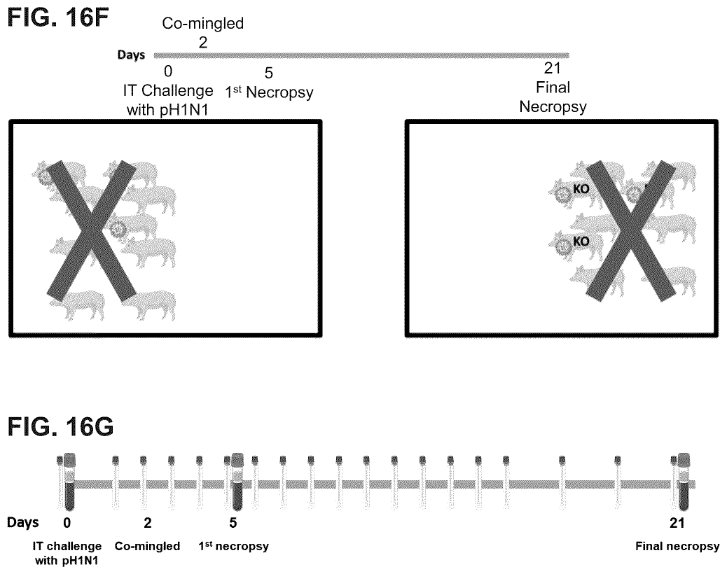

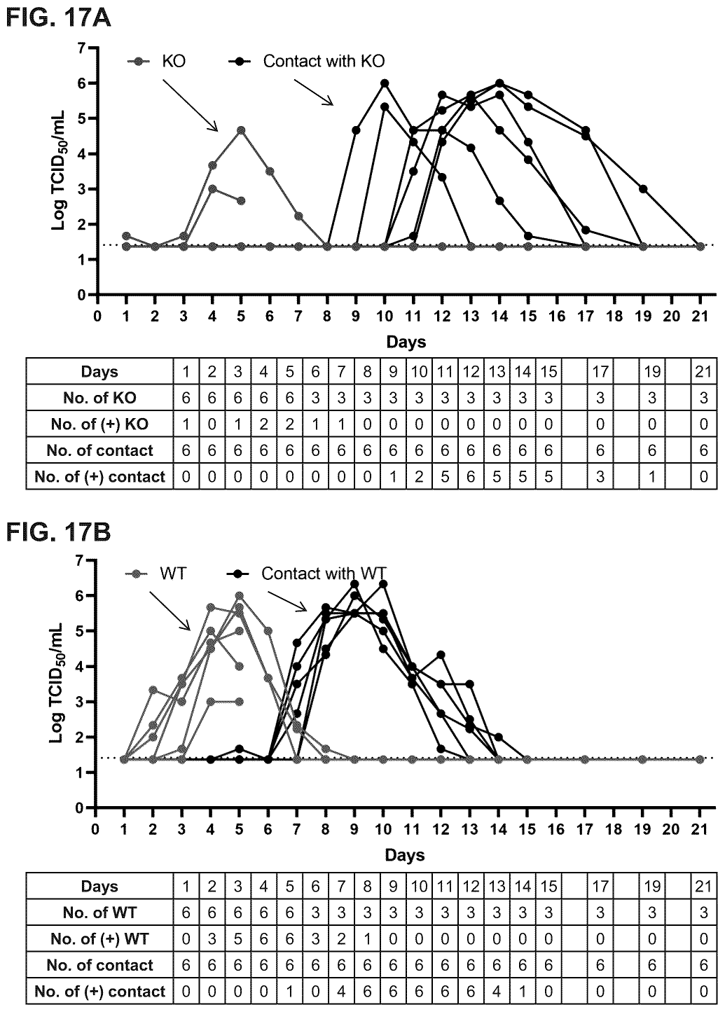

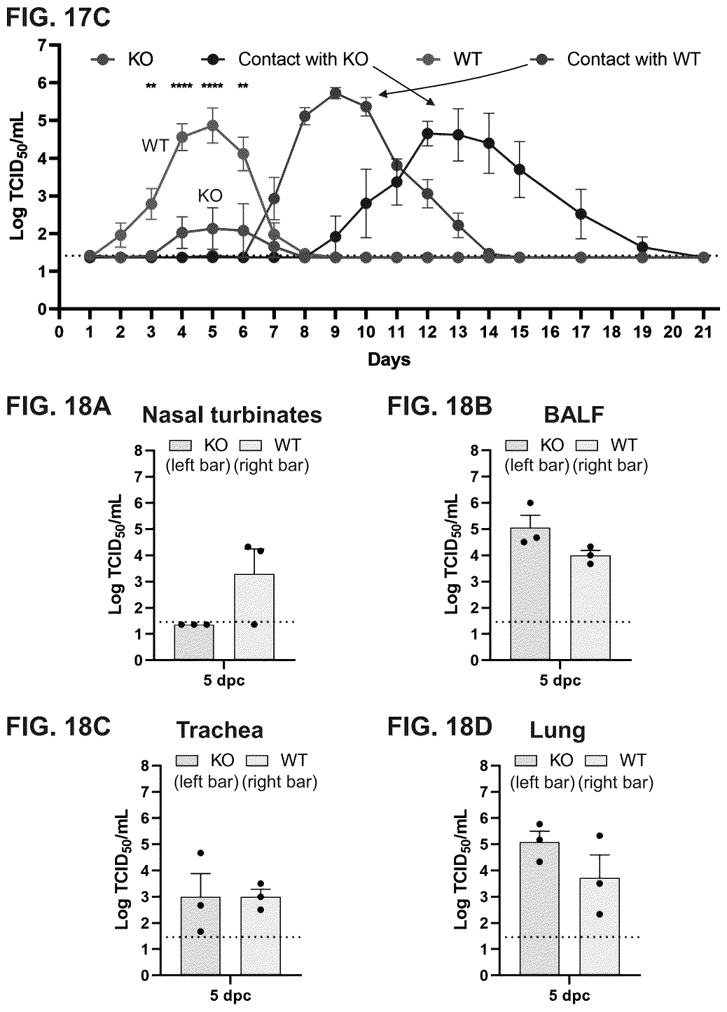

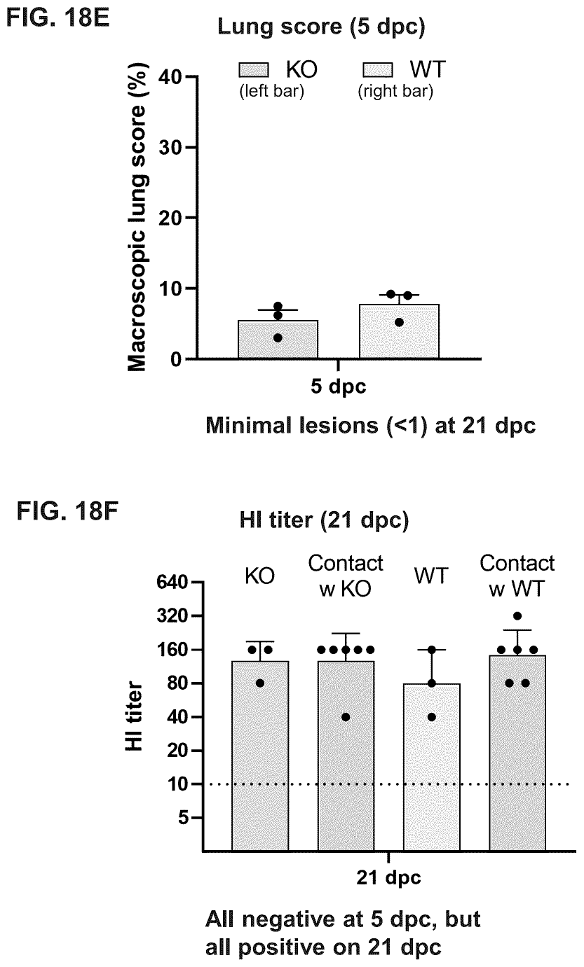

Referring to FIGS. 16A-18F, in the transmission study, a delayed transmission of influenza viruses was found when WT pigs were co-housed with infected KO pigs in comparison to WT pigs co-housed with infected WT pigs.

All contact WT pigs co-housed with infected WT pigs were positive in nasal swabs at 8 days post-challenge (6 days post-co-housing). In contrast, all contact pigs co-housed with KO pigs shed virus in nasal swabs at 12 days post-challenge (10 days post-co-housing).

### Embodiments

The following embodiments are intended to be illustrative only and not to be limiting in any way.

Set A—Knockout Swine. Two or More Modified TMPRSS Genes

Embodiment A1. A knockout swine comprising at least two modified TMPRSS (Transmembrane protease, serine) genes as compared to a swine with wild-type TMPRSS genes, wherein expression of functional gene products of the at least two modified TMPRSS genes in the knockout swine is decreased as compared to a wild-type swine

Embodiment A2. The swine of embodiment 1, wherein the first modified TMPRSS gene is TMPRSS2.

Embodiment A3. The swine of embodiment 1, wherein the second modified TMPRSS gene is TMPRSS4.

Embodiment A4. The swine of embodiment 1, wherein the first modified TMPRSS gene is TMPRSS2 and the second modified TMPRSS gene is TMPRSS4.

Embodiment A5. The swine of embodiment 2 or 4, wherein the wild-type TMPRSS2 gene encodes a transmembrane protease, serine 2 having at least 90% amino acid identity corresponding to an expressed gene of GenBank Gene ID 100739292.

Embodiment A6. The swine of embodiment 2 or 4, wherein the modified TMPRSS2 gene encodes a transmembrane protease, serine 2 having at least 90% amino acid identity corresponding to the wild-type TMPRSS2 gene.

Embodiment A7. The swine of embodiment 3 or 4, wherein the wild-type TMPRSS4 gene encodes a transmembrane protease, serine 4 having at least 90% amino acid identity corresponding to an expressed gene of GenBank Gene ID 100514419.

Embodiment A8. The swine of embodiment 3 or 4, wherein the modified TMPRSS4 gene encodes a transmembrane protease, serine 4 having at least 90% amino acid identity corresponding to the wild-type TMPRSS4 gene.

Embodiment A9. The swine of embodiment 1, wherein each of the two modified TMPRSS genes has at least one disrupted exon.

Embodiment A10. The swine of embodiment 1, wherein one of the modified TMPRSS gene(s) is a TMPRSS2 gene, and wherein the TMPRSS2 gene has a disrupted exon that is any exon other than exon 2.

Embodiment A11. The swine of embodiment 1, wherein one of the modified TMPRSS gene(s) is a TMPRSS4 gene, and wherein the TMPRSS4 gene has a disrupted exon that is any exon other than exon 8.

Embodiment A12. The swine of embodiment 1, wherein one of the modified TMPRSS gene(s) is a TMPRSS4 gene, and wherein the TMPRSS4 gene has a disrupted exon that is any exon other than exon 9.

Set B—Knockout Swine. One Modified TMPRSS Gene

Embodiment B1. A knockout swine comprising a modified TMPRSS (Transmembrane protease, serine) gene as compared to a swine with wild-type TMPRSS genes, wherein expression of functional gene products of the modified TMPRSS gene in the knockout swine is decreased as compared to a wild-type swine.

Embodiment B2. The swine of embodiment 1, wherein the modified TMPRSS gene is TMPRSS2, TMPRSS3, TMPRSS4, TMPRSS5, TMPRSS6, TMPRSS7, TMPRSS9, TMPRSS12, TMPRSS13, or TMPRSS15.

Embodiment B3. The swine of embodiment 1, wherein the wild-type TMPRSS gene is a TMPRSS2 gene that encodes a transmembrane protease, serine 2 having at least 90% amino acid identity corresponding to an expressed gene of GenBank Gene ID 100739292.

Embodiment B4. The swine of embodiment 1, wherein the modified TMPRSS gene is a TMPRSS2 gene that encodes a transmembrane protease, serine 2 having at least 90% amino acid identity corresponding to the wild-type TMPRSS2 gene.

Embodiment B5. The swine of embodiment 1, wherein the wild-type TMPRSS gene is a TMPRSS4 gene that encodes a transmembrane protease, serine 4 having at least 90% amino acid identity corresponding to an expressed gene of GenBank Gene ID 100514419.

Embodiment B6. The swine of embodiment 1, wherein the modified TMPRSS gene is a TMPRSS4 gene that encodes a transmembrane protease, serine 4 having at least 90% amino acid identity corresponding to the wild-type TMPRSS4 gene.

Embodiment B7. The swine of embodiment 1, wherein the modified TMPRSS gene has at least one disrupted exon.

Embodiment B8. The swine of embodiment 1, wherein the modified TMPRSS gene is a TMPRSS2 gene, and wherein the TMPRSS2 gene has a disrupted exon that is any exon other than exon 2.

Embodiment B9. The swine of embodiment 1, wherein the modified TMPRSS gene is a TMPRSS4 gene, and wherein the TMPRSS4 gene has a disrupted exon that is any exon other than exon 8.

Embodiment B10. The swine of embodiment 1, wherein the modified TMPRSS gene is a TMPRSS4 gene, and wherein the TMPRSS4 gene has a disrupted exon that is any exon other than exon 9.

Set C—Knockout Swine. Modified TMPRSS2 Modified at Two or More Separate Regions of TMPRSS2

Embodiment C1. A knockout swine comprising a modified TMPRSS2 (Transmembrane protease, serine 2) gene as compared to a swine with a wild-type TMPRSS2 gene, wherein expression of functional transmembrane protease, serine 2 in the knockout swine is decreased as compared to a wild-type swine and wherein the TMPRSS2 gene is modified at at least two separate regions of the TMPRSS2 gene.

Embodiment C2. The swine of embodiment 1, wherein the modified TMPRSS2 gene has a disrupted Exon 2 and a disrupted Exon 1.

Embodiment C3. The swine of embodiment 1, wherein the modified TMPRSS2 gene has a disrupted Exon 2 and a disrupted Exon 3.

Embodiment C4. The swine of embodiment 1, wherein the wild-type TMPRSS2 gene encodes a transmembrane protease, serine 2 having at least 90% amino acid identity corresponding to an expressed gene of GenBank Gene ID 100739292.

Embodiment C5. The swine of embodiment 1, wherein the modified TMPRSS2 gene encodes a transmembrane protease, serine 2 having at least 90% amino acid identity corresponding to the wild-type TMPRSS2 gene.

Set D—Knockout Swine. Modified TMPRSS2 Modified at any Location Other than Exon 2

Embodiment D1. A knockout swine comprising a modified TMPRSS2 (Transmembrane protease, serine 2) gene as compared to a swine with a wild-type TMPRSS2 gene, wherein expression of functional transmembrane protease, serine 2 in the knockout swine is decreased as compared to a wild-type swine and wherein the modified TMPRSS2 gene has a disrupted exon that is any exon other than Exon 2.

Embodiment D2. The swine of embodiment 1, wherein the TMPRSS2 gene has a disrupted Exon 1.

Embodiment D3. The swine of embodiment 1, wherein the TMPRSS2 gene has a disrupted Exon 3.

Embodiment D4. The swine of embodiment 1, wherein the wild-type TMPRSS2 gene encodes a transmembrane protease, serine 2 having at least 90% amino acid identity corresponding to an expressed gene of GenBank Gene ID 100739292.

Embodiment D5. The swine of embodiment 1, wherein the modified TMPRSS2 gene encodes a transmembrane protease, serine 2 having at least 90% amino acid identity corresponding to the wild-type TMPRSS2 gene.

Set E—Knockout Swine. Modified TMPRSS2 and Modified TMPRSS4

Embodiment E1. A knockout swine comprising a modified TMPRSS2 (Transmembrane protease, serine 2) gene as compared to a swine with wild-type TMPRSS2 gene, and a modified TMPRSS4 (Transmembrane protease, serine 4) gene as compared to a swine with a wild-type TMPRSS4 gene, wherein expression of functional gene products of both the modified TMPRSS2 gene and the modified TMPRSS4 gene in the knockout swine is decreased as compared to a wild-type swine.

Embodiment E2. The swine of embodiment 1, wherein the wild-type TMPRSS2 gene encodes a transmembrane protease, serine 2 having at least 90% amino acid identity corresponding to an expressed gene of GenBank Gene ID 100739292.

Embodiment E3. The swine of embodiment 1, wherein the modified TMPRSS2 gene encodes a transmembrane protease, serine 2 having at least 90% amino acid identity corresponding to the wild-type TMPRSS2 gene.

Embodiment E4. The swine of embodiment 1, wherein the wild-type TMPRSS4 gene encodes a transmembrane protease, serine 4 having at least 90% amino acid identity corresponding to an expressed gene of GenBank Gene ID 100514419.

Embodiment E5. The swine of embodiment 1, wherein the modified TMPRSS4 gene encodes a transmembrane protease, serine 4 having at least 90% amino acid identity corresponding to the wild-type TMPRSS4 gene.

Embodiment E6. The swine of embodiment 1, wherein both the modified TMPRSS2 gene and the modified TMPRSS4 gene each has at least one disrupted exon.

Set F—Method of Producing Pig Zygote. Modified TMPRSS Genes Using Nuclease (Genes Defined by Belonging to Gene Family)

Embodiment F1. A method for producing a genetically modified organism resistant to viral infection, wherein replication of a virus in a cell of the organism is reduced as compared to a wild-type organism, comprising introducing into a zygote of said organism a nuclease comprising a gene-binding moiety, wherein said gene binding moiety is configured to bind at least one gene of said zygote, wherein said one or more genes of said zygote belong to the TMPRSS gene family, its gene family members, its orthologs and paralogs, its isoforms, or any combination thereof, wherein said organism belongs to the family Suidae.

Embodiment F2. The method of embodiment 1, wherein the TMPRSS gene family comprises TMPRSS2 or a fragment thereof, TMPRSS3 or a fragment thereof, TMPRSS4 or a fragment thereof, TMPRSS5 or a fragment thereof, TMPRSS6 or a fragment thereof, TMPRSS7 or a fragment thereof, TMPRSS9 or a fragment thereof, TMPRSS12 or a fragment thereof, TMPRSS13 or a fragment thereof, or TMPRSS15 or a fragment thereof.

Embodiment F3. The method of embodiment 1, wherein said organism is a swine.

Embodiment F4. The method of embodiment 1, where said organism is Sus scrofa.

Embodiment F5. The method of embodiment 1, where said organism is Sus domesticus.

Embodiment F6. The method of embodiment 1, where said organism is Sus scrofa domesticus.

Embodiment F7. The method of embodiment 1, wherein said nuclease is a programmable nuclease comprising at least one of a CRISPR-associated (Cas) polypeptide, a zinc finger nuclease (ZFN), a transcription activator-like effector nuclease (TALEN), or a combination thereof.

In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures.

What is claimed is:

1. A genetically modified transgenic swine comprising at least one modified Transmembrane protease, serine (TMPRSS) gene as compared to a non-genetically modified swine with wild-type TMPRSS gene, wherein expression of functional gene products of the at least one modified TMPRSS gene in the genetically modified transgenic swine is decreased as compared to the non-genetically modified swine.

2. The swine of claim 1, wherein the swine is less susceptible to an influenza virus and sheds significantly less influenza virus as compared to the non-genetically modified swine.

3. The swine of claim 1, wherein the at least one modified TMPRSS gene is TMPRSS2, TMPRSS3, TMPRSS4, TMPRSS5, TMPRSS6, TMPRSS7, TMPRSS9, TMPRSS12, TMPRSS13, or TMPRSS15.

4. The swine of claim 1, wherein the at least one modified TMPRSS gene has at least one disrupted exon.

5. The swine of claim 1, comprising at least two modified TMPRSS genes.

6. The swine of claim 1, wherein the at least one modified TMPRSS gene is a modified TMPRSS2 gene encoding a transmembrane protease, serine 2 having at least 90% amino acid identity corresponding to the wild-type TMPRSS2 gene.

7. The swine of claim 6, wherein TMPRRS2 is knocked out, effectively impairing replication of an influenza virus in the swine as compared to the non-genetically modified swine.

8. The swine of claim 6, wherein the modified TMPRSS2 gene has a disrupted exon that is any exon other than exon 2.

9. The swine of claim 6, wherein the modified TMPRSS2 gene has a disrupted Exon 2 and a disrupted Exon 1.

10. The swine of claim 6, wherein the modified TMPRSS2 gene has a disrupted Exon 2 and a disrupted Exon 3.

11. The swine of claim 1, wherein the at least one modified TMPRSS gene is a TMPRSS4 gene encoding a transmembrane protease, serine 4 gene having at least 90% amino acid identity corresponding to the wild-type TMPRSS4 gene.

12. A method for making a genetically modified transgenic pig, comprising:
a) knocking out (KO) at least one Transmembrane protease, serine (TMPRSS) gene in a pig oocyte;
b) developing the pig oocyte into an embryo;
c) transferring said embryo into a surrogate pig; and
d) gestating in said surrogate pig said embryo into the genetically modified transgenic pig;
wherein expression of functional gene products of the at least one KO TMPRSS gene in the genetically modified transgenic pig is decreased as compared to a non-genetically modified pig.

13. The method of claim 12, wherein the genetically modified transgenic pig is less susceptible to an influenza virus and sheds significantly less influenza virus as compared to the non-genetically modified pig.

14. The method of claim 12, wherein CRISPR/Cas is used to KO the at least one TMPRSS gene.

15. The method of claim 12, wherein at least one KO TMPRSS gene has at least one disrupted exon.

16. The method of claim 12, wherein the at least one KO TMPRSS gene is TMPRSS2, TMPRSS3, TMPRSS4, TMPRSS5, TMPRSS6, TMPRSS7, TMPRSS9, TMPRSS12, TMPRSS13, or TMPRSS15.

17. The method of claim 16, wherein TMPRRS2 is KO, effectively impairing replication of an influenza virus in a cell of the genetically modified transgenic pig as compared to the non-genetically modified pig.

18. The method of claim 17, wherein the KO TMPRSS2 gene has a disrupted exon that is any exon other than exon 2.

19. The method of claim 17, wherein the KO TMPRSS2 gene has a disrupted Exon 2 and a disrupted Exon 1, or a disrupted Exon 2 and a disrupted Exon 3.

20. A genetically modified transgenic pig cell, wherein said genetically modified transgenic pig cell comprises at least one modified Transmembrane protease, serine (TMPRSS) gene, wherein expression of functional gene products of the at least one modified TMPRSS gene in the genetically modified transgenic pig cell is decreased as compared to the non-genetically modified pig cell.
